(19) United States
(12) Patent Application Publication
(10) Pub. No.: US 2020/0279636 A1
(43) Pub. Date: Sep. 3, 2020

(54) SYSTEM FOR PATIENT TRAINING

(71) Applicants: Hillel Maresky, Toronto (CA); Shachar Weis, London (CA)

(72) Inventors: Hillel Maresky, Toronto (CA); Shachar Weis, London (CA)

(21) Appl. No.: 16/781,071

(22) Filed: Feb. 4, 2020

Related U.S. Application Data
(60) Provisional application No. 62/812,593, filed on Mar. 1, 2019.

(51) Int. Cl. G16H 20/70; G06T 19/00; G06T 7/00; G16H 50/20; G16H 30/40; G16H 40/63; G16H 10/60

(52) U.S. Cl. CPC G16H 20/70; G06T 19/006; G06T 7/0012; G16H 50/20; G06T 2207/30004; G16H 40/63; G16H 10/60; G06T 2210/41; G06T 2207/30168; G16H 30/40

(57) ABSTRACT

A system is configured to (A) transmit, to a patient, sensory output signals associated with the virtual medical-imaging system; (B) receive, from the patient, biometric-information signals of the patient that represent a biometric reaction of the patient receiving the sensory output signals associated with the virtual medical-imaging system; and (C) transmit, to the patient, patient-training information configured to urge improvement of a state of calmness of the patient. At least some of the patient-training information takes into account at least some of the biometric-information signals that were received.

### TECHNICAL FIELD

This document relates to the technical field of (and is not limited to) a system for patient training, and more specifically to a computer system configured to train a patient (such as, a medical patient) to have a state of calmness (that is, to remain calm), and/or a method therefor.

### BACKGROUND

There are known methods and/or systems configured to train a person to have calmness, which is the mental state of peace of mind, and/or physical stillness of body, free from agitation, excitement or disturbance. Some disciplines that promote and develop calmness are prayer, yoga, relaxation training, breath training, and meditation.

### SUMMARY

It will be appreciated that there exists a need to mitigate (at least in part) at least one problem associated with the existing systems for training a patient to have a state of calmness (also called the existing technology). After much study of the known systems and methods with experimentation, an understanding (at least in part) of the problem and its solution has been identified (at least in part) and is articulated (at least in part) as follows:

Calmness may also refer to a person being in (having) a state of serenity, tranquillity, stillness or peace (in mind and/or body). Calmness may most easily occur for the average person during a state of relaxation. Calmness may be found during much more alert and aware states of being. Some people find that focusing the mind on something external, or even internal (such as breathing, etc.), may itself be very calming for a person. Calmness is a quality that may be cultivated and increased with practice. It usually takes a trained mind to stay calm in the face of a great deal of different stimulation and possible distractions (especially emotional ones). Negative emotions are the greatest challenge to someone who is attempting to cultivate a calm mind. Another term usually associated with calmness is peace. A mind that is at peace or calm may cause the brain to produce good hormones, which in turn gives the person a stable emotional state and promotes good health (improved health) in other areas of life. It is considered beneficial to stay calm and to cultivate a state of calmness in every possible situation, especially during stressful events.

Known medical-imaging systems are configured to generate a medical image of patients. Sometimes, while a medical-imaging system is generating a medical image of the patient, the patient is not in a calm state of mind. As a result of the restless state of mind (and/or body) of the patient while the medical-imaging system is generating the medical image, the generated medical image may have a less desirable quality (the image may be fuzzy or not in focus with blurred details). Unfortunately, this situation may lead to a less desired outcome for the patient on the basis that the doctor (who is responsible for diagnosing the patient based on the medical image) has a lower possibility for making an accurate (correct) medical diagnosis based on the lower quality of the generated medical image of the patient.

What may be needed is a system or a computer system configured to train a patient to have a state of calmness during the time when the patient receives virtual sensations or sensation signals (via appropriate transducers) that represent sensations associated with a virtual medical-imaging system that is made to appear to be operating (as sensed by the patient in a virtual-reality environment). The virtual-reality environment is set-up and provided by the computer system to the patient.

What may be needed is a system configured to provide training to the patient, and then to evaluate the outcome of the training. The evaluation computation may provide a determination that may be provided by the system. The determination may indicate whether (on a hypothetical basis) the image quality (of the potential medical image to be made of the patient by the real medical-imaging system) may be suitable or good enough for an improved (reliable) medical diagnosis to be made by a medical doctor. This arrangement (system) may avoid wastage of time and effort for the medical doctor in making a relatively more accurate medical diagnosis with improved confidence. After training is completed, the real medical-imaging system may be utilized for generating the real medical image of the patient. The patient may utilize the training for remaining motionless while the real medical image is generated by the real medical-imaging system. On the basis that the patient may remain relatively calm during the generation of the real medical image, the computer system may improve the outcome for the patient. Advantageously, the medical doctor (who is responsible for diagnosing the patient based on the real medical image) has an improved possibility for making a more accurate medical diagnosis based on a better quality of the real generated medical image of the patient. The computer system improves the ability of the medical doctor to make a more accurate medical diagnosis (provided that the quality of the real medical image is as good as possible and that the medical doctor has the necessary skills and awareness for making a good diagnosis).

What may be needed is an apparatus. The apparatus includes, and is not limited to, a system. The system is positionable (is configured to be spatially positioned) relative to a patient (in a spaced-apart relationship). The system configured to electrically connect (directly or indirectly, wired or wirelessly) to output transducers, in which the patient is positionable proximate to the output transducers, and the output transducers are configured to transmit sensory output signals to the patient. The system is also configured to electrically connect (directly or indirectly, wired or wirelessly) to biometric-information sensors (the patient is positionable proximate to the biometric-information sensors, and the biometric-information sensors are configured to receive biometric-information signals from the patient). The system is also configured to transmit, to the patient via the output transducers, (i) sensory output signals associated with a virtual medical-imaging system and (ii) the patient-training information configured to improve an ability of the patient to remain relatively motionless while the patient, in use, receives the sensory output signals. The system is also configured to receive the biometric-information signals (of the patient) via the biometric-information sensors, in which the biometric-information signals represent biometric reactions (of the patient) once the patient reacts to (responds to, during usage of the system) the reception of the sensory output signals. The system is also configured to adapt at least some of the patient-training information based on changes detected in the biometric-information signals received from the patient. Improvement, at least in part, of the ability of the patient to remain relatively motionless provides improvement, at least in part, to the image stability exhibited by a real medical image (to be) generated by a real medical-imaging system as the patient remains motionless relative to the real medical-imaging system while the real medical-imaging system generates the real medical image of the patient. The apparatus may be adapted such that the system is also configured to evaluate an outcome of the patient-training information provided to the patient, and the system is also configured to provide a determination indicating whether the image stability exhibited by the real medical image of the patient, to be generated by the real medical-imaging system, is suitable for an improved medical diagnosis (wastage of time and effort is reduced, at least in part, for the medical doctor in making a relatively more accurate medical diagnosis with improved confidence as a result of the patient utilizing the training for remaining motionless while the real medical image is generated by the real medical-imaging system on the basis that the patient has utilized the patient-training information (the training) configured to train the patient to remain relatively calm while the real medical-imaging system, in use, generates the real medical image (that is, during the generation of the real medical image by the real medical-imaging system).

What may be needed is a computer system configured to train a patient to have a state of calmness (to remain calm or improve the degree of calmness) prior to having the medical-imaging system generate a medical image of the patient. In this manner, the patient has developed an improved state of calmness for when the medical-imaging system is utilized for generating the medical image of the patient in such a way that the generated medical image of the patient has a relatively improved image quality based on the training provided to the patient.

To mitigate, at least in part, at least one problem associated with the existing technology, there is provided (in accordance with an aspect) an apparatus. The apparatus includes a system having an output section configured to transmit, to a patient, sensory output signals associated with the virtual medical-imaging system. The system has an input section configured to receive, from the patient, biometric-information signals of the patient that represent reactions (biometric reactions or a biometric reaction) of the patient receiving the sensory output signals associated with the virtual medical-imaging system. The output section of the system is also configured to transmit, to the patient, patient-training information configured to improve (render) a state of calmness in the mind of the patient. The patient-training information takes into account the biometric-information signals that were received (by the computer system). The state of calmness (of the patient) and the ability (of the patient) to remain relatively motionless may be improved prior to utilization of the real medical-imaging system for generating a real medical image of the patient in such a way that the real medical image of the patient may have a relatively improved image quality based on the training provided by the computer system to the patient (provided that the patient has managed to utilize the training for improving the state of calmness and for remaining relatively motionless while the medical image is generated). The real medical image of the patient is generated by the real medical-imaging system.

What may be needed is an apparatus. The apparatus includes and is not limited to (comprises) a system. The system has an output section that is positionable relative to a patient. The output section is configured to transmit, to the patient, (i) sensory output signals associated with a virtual medical-imaging system, and (ii) patient-training information configured (to be sensed by the patient and) to improve an ability of the patient to remain relatively motionless (while the patient, in use, receives the sensory output signals). The system also has an input section positioned relative to the output section. The input section is configured to receive biometric-information signals from the patient (the biometric-information signals represent biometric reactions of the patient in response to the patient, in use, receiving, and reacting to, the sensory output signals from the output section). At least some of the patient-training information, to be provided by the output section, is adapted based on changes detected in the biometric-information signals received by the input section. Improvement of the ability of the patient to remain relatively motionless provides improvement to image stability of (exhibited by) a real medical image (to be) generated by a real medical-imaging system as the patient remains motionless relative to the real medical-imaging system while the real medical-imaging system generates the real medical image of the patient.

To mitigate, at least in part, at least one problem associated with the existing technology, there is provided (in accordance with an aspect) an apparatus. The apparatus includes a computer system comprising a processor. The processor is configured to electrically connect (directly or indirectly, wired or wirelessly) to biometric-information sensors (the patient is positionable proximate to the biometric-information sensors). The processor is configured to electrically connect (directly or indirectly, wired or wirelessly) to output transducers (the patient is positionable proximate to the output transducers). A memory device tangibly embodies a computer program. The memory device is electrically coupled to the processor. The computer program is configured to be readable and executable by the processor. The computer program is configured to direct the processor to train the patient to have a state of calmness by utilizing the biometric-information sensors and the output transducers (prior to having a real medical-imaging system generate a real medical image of the patient). Advantageously, with this system, the patient may develop an improved state of calmness for when the real medical-imaging system is utilized for generating the real medical image of the patient. This is done in such a way that the generated medical image of the patient has a relatively improved image quality based on the training provided to the patient (that is, if the patient can, in fact, maintain his calmness based on the training that was provided by the computer system).

To mitigate, at least in part, at least one problem associated with the existing technology, there is provided (in accordance with an aspect) an apparatus. The apparatus includes a memory device tangibly embodying a computer program. The memory device is configured to be electrically coupled to a processor of a computer system. The processor is configured to electrically connect (directly or indirectly, wired or wirelessly) to biometric-information sensors. The patient is positionable proximate to the biometric-information sensors. The processor is configured to electrically connect (directly or indirectly, wired or wirelessly) to the output transducers. The patient is positionable proximate to the output transducers. The computer program is configured to be readable and executable by the processor. The computer program is configured to direct the processor to train the patient to have a state of calmness by utilizing the biometric-information sensors and the output transducers (prior to utilizing a real medical-imaging system for generating a real medical image of the patient). The patient utilizes the training (for staying calm and motionless) that was provided by the computer system while the real medical system is generating the medical image of the patient. Advantageously, the system helps the patient to develop an improved state of calmness (an ability for remaining bodily motionless) for when the real medical-imaging system is utilized for generating the real medical image of the patient. This is done in such a way that the generated real medical image of the patient may have a relatively improved image quality based on the training provided to the patient (that is, if the patient can, in fact, utilize the training received from the computer system while the real medical-imaging system is being utilized).

To mitigate, at least in part, at least one problem associated with the existing technology, there is provided (in accordance with an aspect) a method. The method is executable by a computer system. The method includes training the patient to have a state of calmness by utilizing the biometric-information sensors and the output transducers (prior to utilization of a real medical-imaging system for generating a real medical image of the patient).

What may be needed is a method. The method is for improving, at least in part, image of (stability exhibited by) a real medical image (to be) generated by a real medical-imaging system as a patient remains motionless relative to the real medical-imaging system while the real medical-imaging system generates the real medical image of the patient. The method includes and is not limited to (comprises) a transmitting operation (a first operation) including transmitting, to the patient via output transducers, (i) sensory output signals associated with a virtual medical-imaging system, and (ii) patient-training information configured to improve an ability of the patient to remain relatively motionless while the patient, in use, receives the sensory output signals. The method also includes a receiving operation (a second operation) including receiving, from the patient via biometric-information sensors, biometric-information signals representing biometric reactions of the patient in response to the patient, in use, receiving the sensory output signals from the output section. The method also includes an adapting operation (a third operation) including adapting (preferably on the fly adaptation of) at least some of the patient-training information based on changes detected in the biometric-information signals received from the patient. Improvement, at least in part, of the ability of the patient to remain relatively motionless provides improvement, at least in part, to the image stability of (exhibited by) the real medical image (to be) generated by the real medical-imaging system as the patient remains motionless relative to the real medical-imaging system while the real medical-imaging system generates the real medical image of the patient. The method may further include an evaluating operation (a fourth operation) including evaluating (at least in part) an outcome of the patient-training information provided to the patient, and a providing operation (a fifth operation) including providing (at least in part) a determination indicating whether the image stability exhibited by the real medical image of the patient, to be generated by the real medical-imaging system, is suitable for an improved medical diagnosis; in this manner, wastage of time and effort is reduced, at least in part, for the medical doctor in making a relatively more accurate medical diagnosis with improved confidence as a result of the patient has utilizing the patient-training information (the training) for remaining motionless while the real medical image is generated by the real medical-imaging system (on the basis that the patient, in fact, has utilized the patient-training information for remaining relatively calm during the generation of the real medical image by the real medical-imaging system).

To mitigate, at least in part, at least one problem associated with the existing technology, there is provided (in accordance with an aspect) an apparatus. The apparatus includes a computer system comprising a processor. The processor is configured to electrically connect (directly or indirectly, wired or wirelessly) to biometric-information sensors configured to receive biometric-information signals from a patient. The processor is also configured to electrically connect (directly or indirectly, wired or wirelessly) to output transducers configured to transmit, to the patient, sensory output signals associated with a virtual medical-imaging system. A memory device tangibly embodies a computer program. The memory device is electrically coupled (directly or indirectly) to the processor. The computer program is configured to be (directly or indirectly) readable by, and (directly or indirectly) executable by, the processor. The computer program includes program code or coded instructions. The computer program is configured to direct the processor to: (A) transmit, to the patient via the output transducers, the sensory output signals associated with the virtual medical-imaging system; and (B) receive, from the patient (via the biometric-information sensors) the biometric-information signals of the patient that represent reactions (such as, physical motions, movements, reactionary movements, etc.) of the patient while the patient receives the sensory output signals associated with (representing) the virtual medical-imaging system; and (C) transmit, to the patient, via the output transducers, patient-training information (such as, instructions, suggestions, advice, etc.). The patient-training information is configured to help the patient to improve a state of calmness of (such as, the mind and/or body of) the patient (preferably while the patient receives the sensory information from the output transducers). The patient-training information takes into account the biometric-information signals received from the biometric-information sensors. Advantageously, the state of calmness and/or the ability to remain relatively motionless of the patient may be improved (by utilizing the computer system) prior to utilization of a real medical-imaging system for generating a real medical image of the patient. The patient utilizes (to the best of their ability) the training that was provided by the computer system while the real medical image is being generated. This is done in such a way that the real medical image of the patient (which was generated by the real medical-imaging system) may have a relatively improved image quality (as a result of the training provided by the computer system to the patient, and if the patient can manage to utilize that training accordingly).

To mitigate, at least in part, at least one problem associated with the existing technology, there is provided (in accordance with an aspect) an apparatus. The apparatus includes a memory device tangibly embodying a computer program. The memory device is electrically coupled (directly or indirectly) to a processor of a computer system. The processor is configured to electrically connect (directly or indirectly, wired or wirelessly) to biometric-information sensors configured to receive biometric-information signals from a patient. The processor is configured to electrically connect (directly or indirectly, wired or wirelessly) to output transducers configured to transmit sensory output signals associated with a virtual medical-imaging system to the patient. The memory device tangibly embodies a computer program configured to be (directly or indirectly) readable by, and (directly or indirectly) executable by, the processor. The computer program is configured to direct the processor to: (A) transmit, to the patient via the output transducers, the sensory output signals associated with the virtual medical-imaging system; and (B) receive, from the patient (via the biometric-information sensors), the biometric-information signals (of the patient) that represent reactions of the patient receiving the sensory output signals associated with the virtual medical-imaging system; and (C) transmit, to the patient via the output transducers, patient-training information configured to improve a state of calmness of the mind of the patient. The patient-training information takes into account the biometric-information signals received from the biometric-information sensors. Advantageously, the state of calmness and the ability to remain relatively motionless of the patient may be improved prior to utilization of a real medical-imaging system for generating a real medical image of the patient. This is done in such a way that the real medical image of the patient, which was generated by the real medical-imaging system, may have a relatively improved image quality based on the training provided by the computer system to the patient (that is, if the patient can manage to utilize that training accordingly).

To mitigate, at least in part, at least one problem associated with the existing technology, there is provided (in accordance with an aspect) a method. The method includes: operation (A) including transmitting, to the patient via output transducers, sensory output signals associated with a virtual medical-imaging system; and operation (B) including receiving, from the patient via biometric-information sensors, the biometric-information signals of the patient that represent reactions of the patient receiving the sensory output signals associated with the virtual medical-imaging system; and operation (C) including transmitting, to the patient via the output transducers, patient-training information configured to improve the state of calmness of the patient. The patient-training information takes into account the biometric-information signals received from the biometric-information sensors.

Other aspects are identified in the claims. Other aspects and features of the non-limiting embodiments may now become apparent to those skilled in the art upon review of the following detailed description of the non-limiting embodiments with the accompanying drawings. This Summary is provided to introduce concepts in simplified form that are further described below in the Detailed Description. This Summary is not intended to identify potentially key features or possible essential features of the disclosed subject matter, and is not intended to describe each disclosed embodiment or every implementation of the disclosed subject matter. Many other novel advantages, features, and relationships will become apparent as this description proceeds. The figures and the description that follow more particularly exemplify illustrative embodiments.

### DETAILED DESCRIPTION OF THE DRAWINGS

The non-limiting embodiments may be more fully appreciated by reference to the following detailed description of the non-limiting embodiments when taken in conjunction with the accompanying drawings, in which:

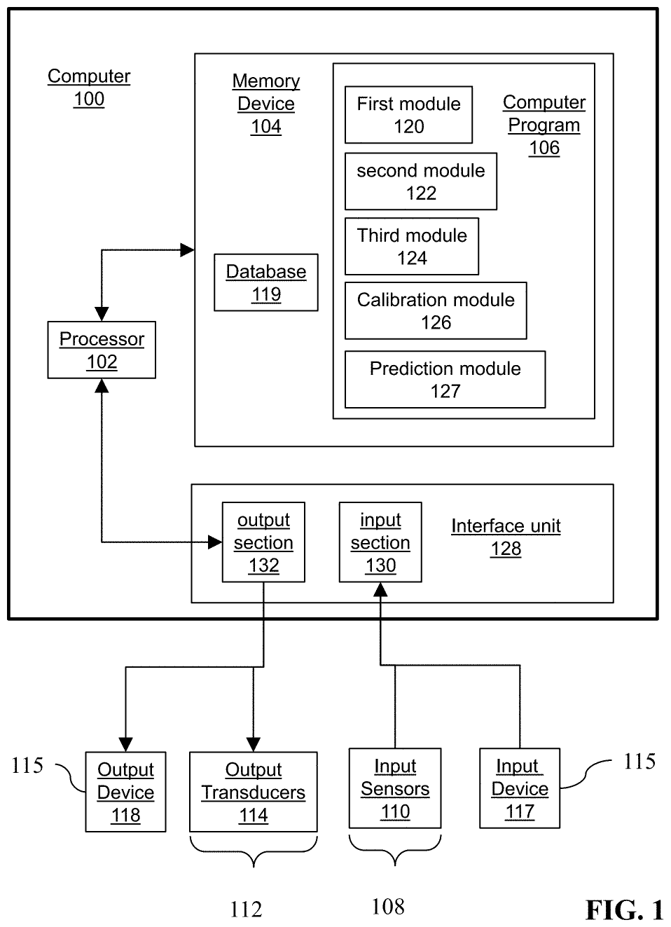

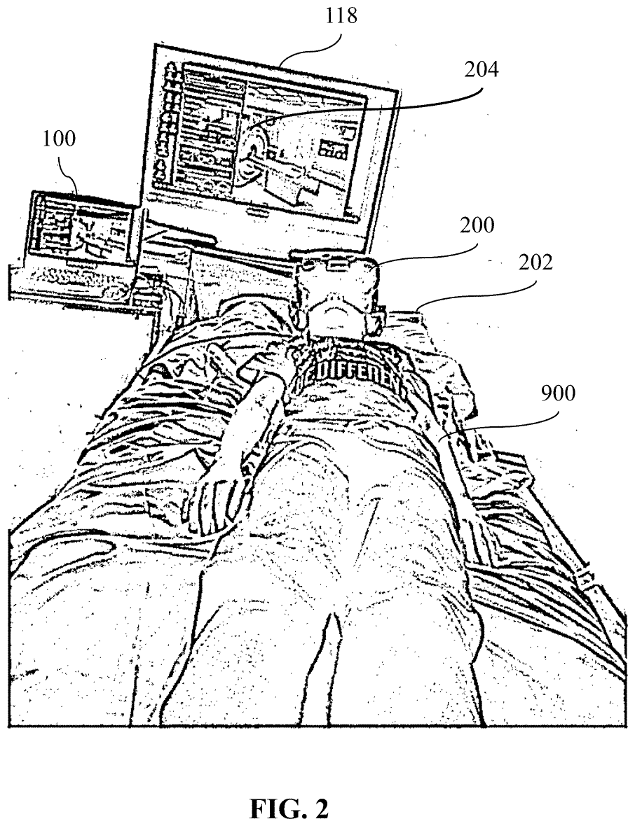

FIG. 1 depicts a schematic view of an embodiment of a computer system configured to train a patient (depicted in FIG. 2) to have a state of calmness prior to having the medical-imaging system generate a medical image of the patient; and

FIG. 2 depicts a front view of an embodiment of a patient that may utilize the computer system of FIG. 1; and

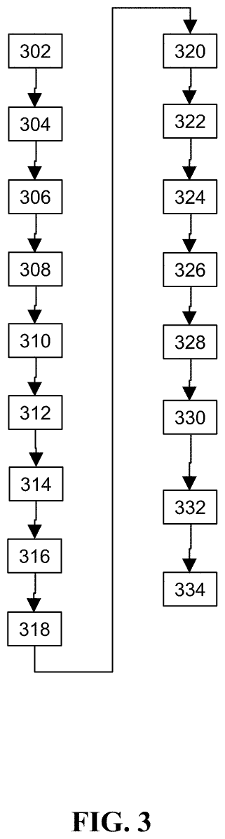

FIG. 3 depicts a schematic view of an embodiment of a flow chart (control logic) to be utilized by the computer system of FIG. 1; and

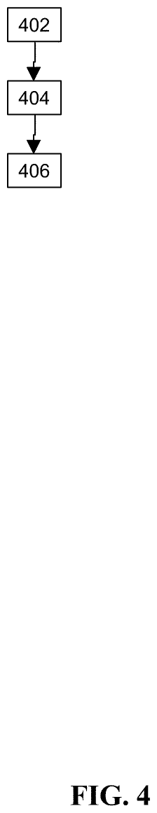

FIG. 4 depicts a schematic view of an embodiment of a flow chart (control logic) to be utilized by the first software module of the computer program to be utilized by the computer system of FIG. 1; and

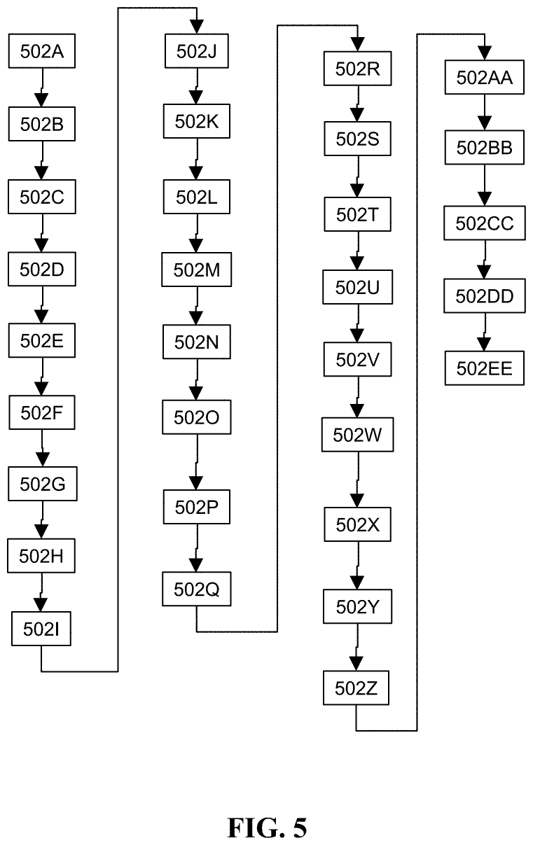

FIG. 5 depicts a schematic view of an embodiment of a flow chart (control logic) to be utilized by the second software module of the computer program to be utilized by the computer system of FIG. 1; and

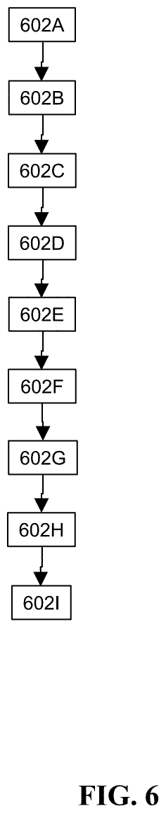

FIG. 6 depicts a schematic view of an embodiment of a flow chart (control logic) to be utilized by the third software module of the computer program to be utilized by the computer system of FIG. 1; and

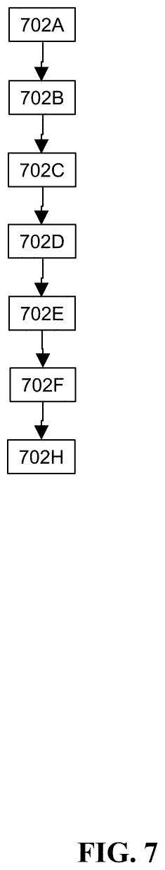

FIG. 7 depicts a schematic view of an embodiment of a flow chart (also called control logic) to be utilized by a calibration software module (software module) of the computer program to be utilized by the computer system of FIG. 1; and

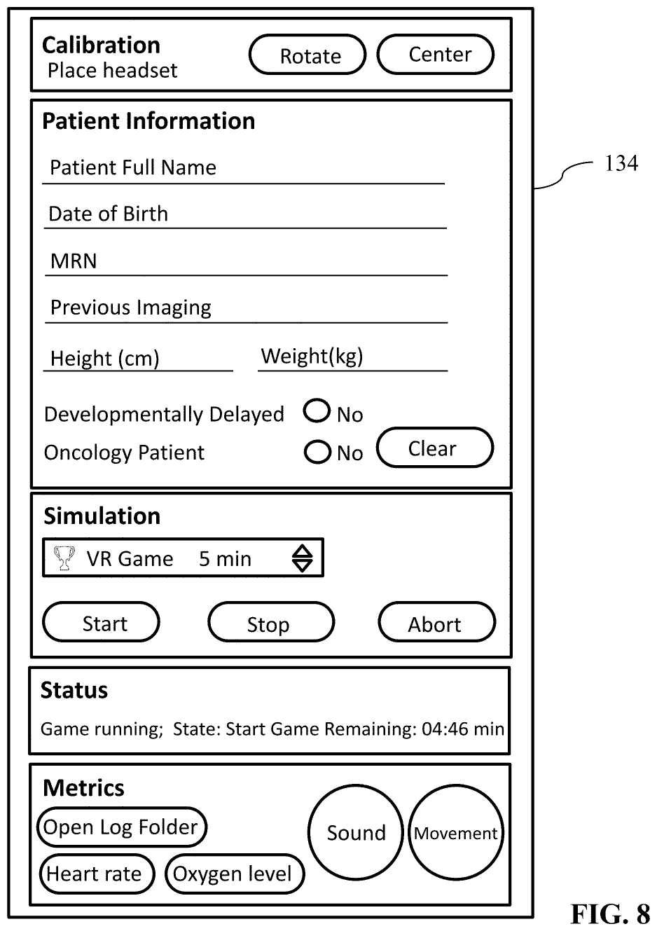

FIG. 8 depicts a schematic view of an embodiment of a user-interface screen to be presented by the computer system of FIG. 1; and

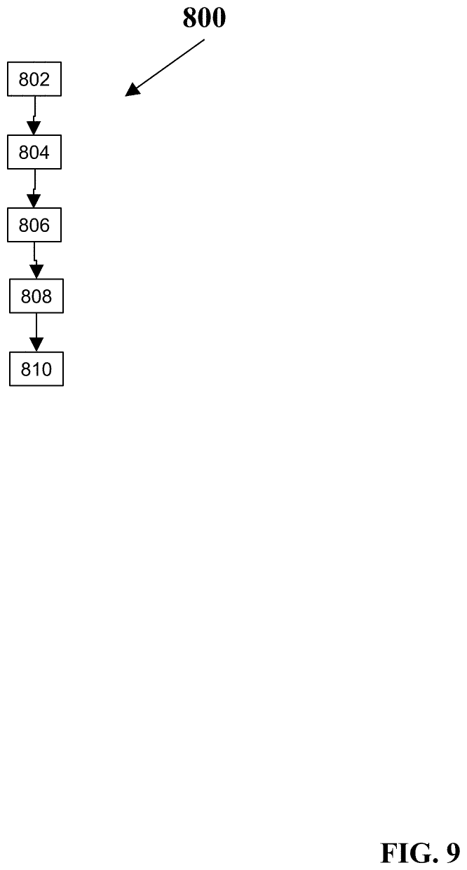

FIG. 9 depicts a schematic view (a flow chart) of a method associated with operations of the computer system of FIG. 1.

The drawings are not necessarily to scale and may be illustrated by phantom lines, diagrammatic representations and fragmentary views. In certain instances, details unnecessary for an understanding of the embodiments (and/or details that render other details difficult to perceive) may have been omitted. Corresponding reference characters indicate corresponding components throughout the several figures of the drawings. Elements in the several figures are illustrated for simplicity and clarity and have not been drawn to scale. The dimensions of some of the elements in the figures may be emphasized relative to other elements for facilitating an understanding of the various disclosed embodiments. In addition, common, and well-understood, elements that are useful in commercially feasible embodiments are often not depicted to provide a less obstructed view of the embodiments of the present disclosure.

LISTING OF REFERENCE NUMERALS
USED IN THE DRAWINGS

100 system (or computer system)
102 processor
104 memory device
106 computer program
108 input collection
110 biometric-information sensors
112 output collection
114 output transducers
115 user interface
117 input device
118 display device
119 database
120 first software module
122 second software module
124 third software module
126 calibration software module
127 prediction module
128 interface unit
130 input section
132 output section
200 virtual-reality headset
202 real bed
204 virtual image
302 first operation
304 second operation
306 third operation
308 fourth operation
310 fifth operation
312 sixth operation
314 seventh operation
316 eighth operation
318 ninth operation
320 tenth operation
322 eleventh operation
324 twelfth operation
326 thirteenth operation
328 fourteenth operation
330 fifteenth operation
332 sixteenth operation
334 seventeenth operation
402 operation
404 execute movement operations
406 recording operation
502A first game-delivery operation
502B second game-delivery operation
502C third game-delivery operation
502D fourth game-delivery operation
502E fifth game-delivery operation
502F sixth game-delivery operation
502G seventh game-delivery operation
502H eighth game-delivery operation
502I ninth game-delivery operation
502J tenth game-delivery operation
502K eleventh game-delivery operation
502L twelfth game-delivery operation
502M thirteenth game-delivery operation
502N fourteenth game-delivery operation
502O fifteenth game-delivery operation
502P sixteenth game-delivery operation
502Q seventeenth game-delivery operation
502R eighteenth game-delivery operation
502S nineteenth game-delivery operation
502T twentieth game-delivery operation
502U twenty-first game-delivery operation
502V twenty-second game-delivery operation
502W twenty-third game-delivery operation
502X twenty-fourth game-delivery operation
502Y twenty-fifth game-delivery operation
502Z twenty-sixth game-delivery operation
502AA twenty-seventh game-delivery operation
502BB twenty-eighth game-delivery operation
502CC twenty-ninth game-delivery operation
502DD thirtieth game-delivery operation
502EE thirty-first game-delivery operation
602A first anxiety-relief training operation
602B second anxiety-relief training operation
602C third anxiety-relief training operation
602D fourth anxiety-relief training operation
602E fifth anxiety-relief training operation
602F sixth anxiety-relief training operation
602G seventh anxiety-relief training operation
602H eighth anxiety-relief training operation
602I ninth anxiety-relief training operation
702 calibration operation
702A first calibration operation
702B second calibration operation
702C third calibration operation
702D fourth calibration operation
702E fifth calibration operation
702F sixth calibration operation
702G seventh calibration operation
702H eighth calibration operation
800 method
802 transmit operation
804 receive operation
806 adaptation operation
808 evaluation operation
810 provision operation
900 patient

### DETAILED DESCRIPTION OF THE NON-LIMITING EMBODIMENT(S)

The following detailed description is merely exemplary and is not intended to limit the described embodiments or the application and uses of the described embodiments. As used, the words “exemplary” or “illustrative” mean “serving as an example, instance, or illustration.” Any implementation described as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure. The scope of the claim is defined by the claims (the claims may be amended during patent examination after the filing of this application). For the description, the terms “upper,” “lower,” “left,” “rear,” “right,” “front,” “vertical,” “horizontal,” and derivatives thereof shall relate to the examples as oriented in the drawings. There is no intention to be bound by any expressed or implied theory in the preceding Technical Field, Background, Summary or the following detailed description. It is also to be understood that the devices and processes illustrated in the attached drawings, and described in the following specification, are exemplary embodiments (examples), aspects and/or concepts defined in the appended claims. Hence, dimensions and other physical characteristics relating to the embodiments disclosed are not to be considered as limiting, unless the claims expressly state otherwise. It is understood that the phrase “at least one” is equivalent to “a”. The aspects (examples, alterations, modifications, options, variations, embodiments and any equivalent thereof) are described regarding the drawings. It should be understood that the invention is limited to the subject matter provided by the claims, and that the invention is not limited to the particular aspects depicted and described. It will be appreciated that the scope of the meaning of a device configured to be coupled to an item (that is, to be connected to, to interact with the item, etc.) is to be interpreted as the device configured to be coupled to the item, either directly or indirectly. Therefore, “configured to” may include the meaning “either directly or indirectly” unless specifically stated otherwise.

FIG. 1 depicts a schematic view of an embodiment of a computer system 100 configured to train a patient 900 (depicted in FIG. 2) to have a state of calmness prior to having the medical-imaging system generate a medical image of the patient 900.

FIG. 2 depicts a front view of an embodiment of a patient 900 that may utilize the computer system 100 of FIG. 1.

Referring to the embodiments as depicted in FIG. 1 and FIG. 2, the computer system 100 is configured to train a patient to have a state of calmness (and/or to remain motionless for a predetermined period of time). Calmness is the state or quality of being free from physical and/or mental agitation or strong emotion. More specifically, the computer system 100 (depicted in FIG. 1 and FIG. 2) is configured to train a patient 900 (depicted in FIG. 2) to have a state of calmness (leading to a state of without movement or motionless, for at least a predetermined period of time). After the training is completed, the patient may use the training to invoke a state of calmness (to the best of their ability) during a time when a real medical-imaging system (known and not depicted) is utilized for generating a real medical image of the patient 900. On the basis that the patient 900 may utilize the training for remaining relatively calm during the generation of the real medical image, the computer system 100 may improve the outcome for the patient 900 on the basis that a doctor (who is responsible for diagnosing the patient 900 based on the real medical image) has an improved possibility for making a better (proper) medical diagnosis based on a better quality version of the generated medical image of the patient 900. Preferably, the computer system 100 is configured to train the patient 900 to have a state of calmness (to remain calm or improve the degree of calmness) prior to having the real medical-imaging system generate a real medical image of the patient 900; in this manner, the patient 900 may develop an ability or skills to invoke an improved state of calmness while the real medical-imaging system is utilized for generating the real medical image of the patient 900. The generated medical image of the patient 900 may have a relatively improved image quality based on the training provided to the patient 900 by the computer system 100 (and based on whether the patient 900 utilizes any of the training that was provided).

Referring to the embodiments as depicted in FIG. 1 and FIG. 2, the computer system 100 of FIG. 1 is configured to train the mind of the patient 900 of FIG. 2 to, preferably, remain in a relatively calm state (and/or a relatively physically motionless state) in response to the senses of the patient 900 who receives signals of information (the outward expressions of a virtual medical-imaging system, such as visual information, auditory information, haptic information, etc.) associated with a virtual medical-imaging system. Haptic technology or kinesthetic communication recreates the sense of touch by applying forces, vibrations, or motions to the user. This mechanical stimulation can be used to assist in the creation of virtual objects in a computer simulation (stimulation), to control such virtual objects, and to enhance the remote control of machines and devices. Haptic devices may incorporate tactile sensors that measure forces exerted by the user on the interface. Preferably, the computer system 100 is a virtual-training system or a virtual reality-based training system. Virtual training refers to training done in a virtual or simulated environment. Virtual training is an interactive and immersive teaching method and/or system that includes technology to provide virtual scenarios to simulate situations that might occur in actual settings, and the training is conducted in a safe, controlled and forgiving environment. For the case where the mind of the patient 900 may be trained by the computer system 100 to remain relatively calm under these circumstances (while the patient 900 receives the training), the patient 900 may be more able to maintain his body (and/or mind) in a calm state or a motionless state of being while the senses of the patient 900 are exposed to (receive) the outward expressions (sensations) of virtual-reality sensations of the virtual medical-imaging system. For the case where the body of the patient 900 receives the training from the computer system 100, the medical images to be generated by the real medical-imaging system may exhibit better image quality thereby improving medical diagnostic examination of the medical images provided by the real medical-imaging system.

Referring to the embodiment as depicted in FIG. 1, the computer system 100 (specifically, the processor 102) is configured to electrically connect (directly or indirectly, wired or wirelessly) to an input collection 108 of the biometric-information sensors 110 (also called input sensors). The biometric-information sensors 110 are configured to provide biometric-information signals of the patient 900. The patient 900 (of FIG. 2) is positioned proximate to the biometric-information sensors 110, such as for the case where the patient 900 is positioned horizontally on a real bed surface (as depicted in FIG. 2). Preferably, the computer system 100 includes an input section 130 configured to electrically connect (directly or indirectly, wired or wirelessly) to the input collection 108. Each of the biometric-information sensors 110 is configured to provide a physiological signal (to the input section of the computer system 100). Each physiological signal corresponds with a respective physiological aspect of the patient 900 of FIG. 2. In accordance with a preferred embodiment, the input collection 108 of the biometric-information sensors 110 includes (and is not limited to): (A) a first input sensor (such as, a heart rate sensor); (B) a second input sensor (such as, a blood pressure sensor); (C) a third input sensor (such as, an oxygen sensor); (D) a fourth input sensor (such as, a microphone for receiving audio emitted from the patient 900); and (E) a fifth input sensor (such as, a motion sensor for detection of motion of the patient 900). It will be appreciated that any combination of the input sensors may be installed onto a platform or housing (if desired). For instance, a virtual-reality headset 200 (depicted in FIG. 2) may include a combination of the biometric-information sensors 110. The processor 102 may include at least one or more processing units (CPU or central processing units), etc. The biometric-information sensors 110 include any one of (or more of, in any combination and/or permutation thereof) a heart rate sensor configured to sense a heart signal associated with the patient 900; a blood pressure sensor configured to sense a blood pressure signal associated with the patient 900; an oxygen sensor configured to sense a flow of oxygen associated with (into and/or out from) the patient 900; a microphone configured to sense audio emitted from the patient 900; and/or a motion sensor configured to sense motion of the patient 900. Preferably, at least one of the biometric-information sensors 110 is selected from the group consisting of a heart rate sensor, a blood pressure sensor, an oxygen sensor, and a microphone.

Referring to the embodiment as depicted in FIG. 1, the computer system 100 (specifically, the processor 102) is configured to electrically connect (directly or indirectly, wired or wirelessly) to an output collection 112 of the output transducers 114. Preferably, the computer system 100 includes an output section 132 configured to electrically connect (directly or indirectly, wired or wirelessly) to the output collection 112. The patient 900 (of FIG. 2) is positioned proximate to the output transducers 114, such as for the case where the patient 900 is positioned horizontally on a real bed surface (depicted in FIG. 2). In accordance with a preferred embodiment, the output collection 112 of the output transducers 114 includes (and is not limited to): (A) a first output transducer, such as a speaker for transmitting audio signals to the ears of the patient 900; (B) a second output transducer, such as a display device or virtual-reality goggles or virtual-reality headset, for transmitting visual images to the eyes of the patient 900; and (C) a third output transducer, such as a haptic device for transmitting tactile sensations (a vibration motion) to the body of the patient 900. The output transducers 114 include any one of (or in any combination and/or permutation thereof): a speaker configured to transmit audio signals to the ears of the patient 900; a display device configured to transmit visual image signals to the eyes of the patient 900; and/or a haptic device configured to transmit tactile sensation signals to the body of the patient 900. At least one of the output transducers 114 is selected from the group consisting of a speaker, a display device, and a haptic device.

Referring to the embodiment as depicted in FIG. 1, the input section 130 (also called an input interface) and/or the output section 132 (also called an output interface) are collectively called the interface unit 128 (also called an interface section). The input section 130 and/or the output section 132 is configured to interface a device (also called an I/O device, an input device such as the biometric-information sensors 110, or an output device such as the output transducers 114, etc.) with the processor 102. The processor 102 may communicate with device via a bus (known and not depicted). The interface unit 128 (the input section 130 and the output section 132) may have control logic and/or components configured to interpret the device address generated by the processor 102. Handshaking may be implemented by the interface unit 128 by using appropriate commands (like BUSY, READY, and WAIT). The processor 102 may communicate with the I/O devices through the interface unit 128. If different data formats are being exchanged, the interface unit 128 is configured to convert serial data to parallel form and vice versa. Because it would be a waste for the processor 102 to be idle while the processor 102 waits for data from an input device there may be provision for generating interrupts and the corresponding type numbers for further processing by the processor 102 if required. For instance, the computer system 100 may use memory-mapped I/O accesses hardware by reading and writing to specific memory locations, may use the same assembly language instructions that the processor 102 may normally use to access memory. An alternative method is via instruction-based I/O which may require that the processor 102 have specialised instructions for input and/or output operations. Both input and output devices have a data processing rate that may vary. With some devices able to exchange data at very high speeds direct access to memory (DMA) without the continuous aid of the processor 102 may be required. The organization of the interface unit 128 (also called the I/O subsystem, the interface section, or a combination of the input section 130 and the output section 132) of the computer system 100 may depend upon the size of the computer system 100 and the peripherals connected to the computer system 100. The interface unit 128 of the computer system 100 may provide an efficient mode of communication between the computer system 100 and the outside environment of the computer system 100. For instance, embodiment of the input devices and output devices may include a monitor (display), a keyboard, a mouse, a printer, etc. The devices that are under the direct control of the computer system 100 are said to be connected online. The interface unit 128 (I/O interface) is configured to transfer information between internal storage (also called the memory device 104) of the computer system 100 and the external devices (also called peripherals, I/O devices, such as the output transducers 114 and the biometric-information sensors 110, etc.). The external devices connected to the computer system 100 may need a communication links for interfacing the external devices with the processor 102. The communication link is configured to resolve the differences (engineering specifications) that exist between the processor 102 and each of the external devices. To resolve these differences, the computer system 100 may include hardware components (placed between the processor 102 and the external devices) configured to supervise and synchronize the input and out transfers (transfers of signals or information). The hardware components may be called interface units because they are configured to interface between the processor bus (of the processor 102) and the external devices (also called peripheral devices).

Referring to the embodiment as depicted in FIG. 2, the display device 118 depicts an embodiment of a virtual image 204 (virtual-reality image or virtual stimulation) of a virtual medical imaging system that is presented during the training session (presented by the computer system 100) to the patient 900 via the virtual-reality headset 200 (which is an embodiment of at least one of the output transducers 114, as depicted in FIG. 1).

Referring to the embodiments as depicted in FIG. 1 and FIG. 2, the computer system 100 trains the mind of the patient 900 to (A) become more calm (to improve the level of calmness and/or the ability to remain physically motionless for a predetermined period of time) while the computer system 100 provides information associated with the virtual image 204 (of the virtual medical-imaging system) to the patient 900, and (B) become less fidgety (less inclined to fidget, less restless, less uneasy, less nervous) while the computer system 100 provides (presents), via at least one of the output transducers 114, the information associated with the virtual image 204 to the patient 900. Calmness is the mental state of peace of mind (being free from agitation, excitement or disturbance). It also refers to being in a state of serenity, tranquillity or peace. The negative emotions (stressed emotions, distracted emotions, bad feelings, etc.) are the greatest challenge to a patient 900 who is attempting to cultivate a calm mind and/or body.

Referring to the embodiments as depicted in FIG. 1 and FIG. 2, the computer system 100 may provide, or facilitate, an improved outcome for obtaining better quality medical images of the patient 900 from a real medical-imaging system (after the training of the patient 900 is completed to an appropriate or sufficient degree). The computer system 100 may include program code configured to assess the potential medical-image quality based on the training provided to the patient 900 and based on the progress made by the patient 900 with the training provided. For the case where the patient 900 receives sensory-training information (also called virtual-reality information from the computer system 100) associated with a virtual medical-imaging system, the real medical-imaging system may provide better-quality medical images of the patient 900 (on the basis that the patient 900 is able to utilize the training for remaining relatively motionless while the patient 900 is positioned proximate to the real medical-imaging system). It will be appreciated that the virtual version or the real version of the medical-imaging system may include (and would not be limited to) an MRI scanner (Magnetic Resonance Imaging scanner or system), a CT scanner (computerized tomography scanner), US scanner (Ultra Sound scanner), etc. For instance, the training information may include virtual-reality training information. For instance, the virtual-reality training information may include stimulation (simulation) information (visual, tactile, audio, etc.) associated with (of) the virtual medical-imaging system.

Referring to the embodiment as depicted in FIG. 1, the computer system 100 is any electronic system configured to store and process data (information), such as in a binary form (preferably, according to instructions of a program). The computer system 100 includes a processor 102. The computer system 100 also includes a memory device 104. The memory device 104 has (tangibly embodies or tangibly stores) a computer program 106 (therein or thereon). The computer program 106 may be referred to as software. The memory device 104 is electrically coupled (either directly or indirectly) to the processor 102 (via a bus, which is known and not depicted). The computer program 106 is configured to be executable by the processor 102. The computer program 106 includes computer code (computer instructions, software) configured to be read by, and executed by, the processor 102, and, in response, the processor 102 operates in accordance with instructions provided by the computer program 106. The processor 102 is configured to read the computer program 106 in response to the processor 102 being directed accordingly. The processor 102 is configured to execute the computer program 106 once the processor 102 reads (receives) the computer program 106. The computer program 106 is configured to instruct (urge) the processor 102 to execute (perform) preprogrammed operations on or with components (such components may include sensors, transducers, input modules, output modules, units, modules, etc.) of the computer system 100 (whether these components are positioned internally to, or externally of, the computer system 100). The computer program 106 is configured to instruct (urge) the processor 102 to interact (operatively interact) with the components (units, modules, etc.) of the computer system 100 in a predetermined and repeatable manner.

Referring to the embodiment as depicted in FIG. 1, the processor 102 may include one or more processor units, such as a digital signal processor configured to perform or execute digital signal processing operations. A digital signal processor (DSP) is a specialized microprocessor (or a SIP block), with its architecture optimized for the operational needs of digital signal processing. The DSP is configured to measure, filter or compress continuous real-world analog signals. The processor 102 may also execute digital signal processing algorithms successfully, but may not be able to keep up with such processing continuously in real-time. The DSP may have a better power efficiency, thus may be more suitable. Digital signal processing is the use of digital processing, such as by the computer system 100, to perform a wide variety of signal processing operations. The signals processed in this manner are a sequence of numbers that represent samples of a continuous variable in a domain such as time, space, or frequency. Digital signal processing and analog signal processing are subfields of signal processing. DSP applications include audio and speech processing and/or other sensor array processing, spectral density estimation, statistical signal processing, digital image processing, signal processing for biomedical engineering, etc. DSP is applicable to both streaming data and static (stored) data.

Referring to the embodiment as depicted in FIG. 1, the computer program 106, preferably, includes coded instructions (computer-programmed coded instructions) configured to be readable by, and executable by, the processor 102. Equivalents to the computer program 106 include (and are not limited to): (A) machine-language code, (B) assembly-language code, and/or (C) source code formed in a high-level computing language (such as C+++, etc.) understood by humans. The high-level language of the source code is compiled into either an executable machine code file or a non-executable machine-code object file (as known by those skilled in the art). Other equivalents to the computer program 106 may include: (A) an application-specific integrated circuit and any equivalent thereof, and/or (B) a field-programmable gate array (FPGA) and any equivalent thereof. It will be appreciated that the computer program 106 includes operational steps to direct the processor 102 to compute (provide computing functions, such as calculations, comparisons, etc.). The computer program 106 is configured to urge the processor 102 to perform predetermined computer operations (depicted as flow charts or control logic), shown in any one of the embodiments of FIG. 3, FIG. 4, FIG. 5, FIG. 6 and/or FIG. 7. It will be appreciated that computing hardware and other operating components are utilized and are suitable for performing the computing processes of the embodiments and are not intended to limit the applicable environments. One of skill in the art will immediately appreciate that the embodiments may be practiced with other computer system configurations, including set-top boxes, hand-held devices, multiprocessor systems, microprocessor-based or programmable consumer electronics, network computers, minicomputers, mainframe computers, and the like. The processor 102 may include a conventional microprocessor such as the INTEL (TRADEMARK) PENTIUM (TRADEMARK of INTEL) microprocessor or the MOTOROLA (TRADEMARK) POWER PC (TRADEMARK of MOTOROLA) microprocessor. One of skill in the art will immediately appreciate that the embodiments of the processor 102 may be practiced with other configurations, including set-top boxes, hand-held devices, multiprocessor systems, microprocessor-based or programmable consumer electronics, network computers, minicomputers, mainframe computers, and the like, etc. One of skill in the art will immediately recognize that the memory device 104 (also called a computer-readable medium or a machine-readable medium, etc.) may include any type of storage device that is accessible by the processor 102 or by other data processing systems. The memory device 104 may be embodied on a magnetic hard disk or an optical disk having executable instructions to cause the processor 102 to perform a computing method (operational steps or computing operations, etc.). Computer hardware (operating components and any equivalent thereof) suitable for performing the processes of the embodiments are not intended to limit the applicable environments.

Referring to the embodiment as depicted in FIG. 1, the computer program 106 is configured to direct the processor 102 to train the patient 900 to have (improve) a state of calmness (of the mind of the patient 900) by utilizing signals (including virtual-reality signals of a virtual medical-imaging system) associated with the biometric-information sensors 110 and the output transducers 114. After the training is completed, the computer system 100 may be utilized to estimate the potential quality of a medical image that may be generated by a real medical-imaging system on the basis of the training provided to the patient 900 and on the basis of the progress made by the patient 900 during the training sessions. On the basis that the computer system 100 may indicate that the patient 900 is potentially ready, then a real medical-imaging system may be utilized for generating a real medical image of the patient 900 (with an improved outcome for the image quality of the medical image to be generated). In this manner, the patient 900 has developed an improved state of calmness for when the real medical-imaging system is utilized for generating the real medical image of the patient 900, and the generated medical image of the patient 900 may have a relatively improved image quality based on the training provided to the patient 900 (provided the patient 900 manages to utilize such training while the medical image is being generated).

Referring to the embodiment as depicted in FIG. 1 the computer program 106 (also called the software program) includes at least (A) a first software module 120 (which is depicted in FIG. 4, and is also called a first program module); (B) a second software module 122 (which is depicted in FIG. 5, and is also called a second program module); (C) a third software module 124 (which is depicted in FIG. 6, and is also called a third program module); and (D) a calibration software module 126 (which is depicted in FIG. 6, and is also called a fourth program module). A software module includes a block of computer-executable instructions or code that can be invoked by the processor 102 in the way that a procedure, function, or method may be invoked (by the processor 102).

Referring to the embodiment as depicted in FIG. 1 and FIG. 2, there is depicted (in general terms) a computer system 100 including a processor 102 configured to electrically connect (directly or indirectly, wired or wirelessly) to biometric-information sensors 110 configured to receive biometric-information signals from a patient 900. The processor 102 is also configured to electrically connect (directly or indirectly, wired or wirelessly) to output transducers 114 configured to transmit sensory output signals associated with a virtual medical-imaging system to the patient 900. A memory device 104 tangibly embodies a computer program 106. The memory device 104 is electrically coupled (directly or indirectly) to the processor 102. The computer program 106 is configured to be (directly or indirectly) readable by, and (directly or indirectly) executable by, the processor 102. The computer program 106 is configured to direct the processor 102 to: (A) transmit, to the patient 900 via the output transducers 114, the sensory output signals associated with the virtual medical-imaging system; and (B) receive, from the patient 900 via the biometric-information sensors 110, the biometric-information signals of the patient 900 that represent reactions (such as, biometric reactions, a biometric reaction, etc.) of the patient 900 receiving the sensory output signals associated with the virtual medical-imaging system; and (C) transmit, to the patient 900 via the output transducers 114, patient-training information configured to improve a state of calmness of the mind of the patient 900. The patient-training information is configured to help the patient 900 to improve a state of calmness of the mind and/or body of the patient 900 (while the patient 900 receives the sensory information from the output transducers 114). The patient-training information takes into account the biometric-information signals received from the biometric-information sensors 110. Advantageously, the state of calmness and the ability to remain relatively motionless of the patient 900 may improve (by utilizing the computer system 100) prior to the utilization of the real medical-imaging system for generating the real medical image of the patient 900. The real medical image of the patient 900, which was generated by the real medical-imaging system, may have a relatively improved image quality based on the training provided by the computer system 100 to the patient 900 that permits or enhances (at least in part) the improvement of the state of calmness and/or the ability to remain relatively motionless for the patient 900.

FIG. 3 depicts a schematic view (representation) of an embodiment of a flow chart (control logic) to be utilized by (executed by) the processor 102 of the computer system 100 of FIG. 1.

Referring to the embodiment as depicted in FIG. 1, the computer system 100 includes an interface unit 128 having an input section 130 (configured to receive input signals), and an output section 132 (configured to provide or transmit output signals). The input section 130 is configured to interface (directly or indirectly) the processor 102 with the input devices as depicted in FIG. 1. The output section 132 is configured to interface (directly or indirectly) the processor 102 with the output devices as depicted in FIG. 1. It will be appreciated that persons skilled in the art would know how to configure (arrange) the input section 130 and the output section 132 depending on the specifications for the types of input signals and output signals, etc.

Referring to the embodiment as depicted in FIG. 1 and FIG. 2, there is depicted (in general terms) a computer system 100 (or simply referred to as a system) including (and not limited to) an output section 132 configured to transmit, to a patient 900, sensory output signals associated with a virtual medical-imaging system. The system also includes an input section 130 configured to receive, from the patient 900, biometric-information signals of the patient 900 that represent reactions of the patient 900 receiving the sensory output signals associated with the virtual medical-imaging system. The output section 132 is also configured to transmit, to the patient 900, patient-training information configured to improve a state of calmness of the patient 900 (in mind and/or body). The patient-training information takes into account (is adjusted to take into account) aspects of the biometric-information signals that were received.

Referring to the embodiment as depicted in FIG. 1 and FIG. 3, the computer program 106 is configured to instruct the processor 102 to execute a first operation 302 (a read operation), including receiving (reading) the signals from the input collection 108 of the biometric-information sensors 110 (so that the biometric information of the patient 900 may be sent to the computer system 100, for processing and/or storage, etc.).

Referring to the embodiment as depicted in FIG. 1 and FIG. 3, the computer program 106 is configured to instruct the processor 102 to execute a second operation 304, including transmitting (writing, sending) sensory output signals (also called sense-stimulation signals) to at least one of the output transducers 114. This is done in such a way that the patient 900 may receive a detectable sensory sensation (sensation signal) associated with (representing an aspect of) the virtual medical-imaging system. The sensory output signals may include the sensations (such as audio information, noise, texture, visual data, and/or tactile data) associated with a virtual medical-imaging system (or a real medical-imaging system). The sense-stimulation signals are associated with the operations of (aspects of) the virtual medical-imaging system. The sense stimulation signals are stored in the memory device 104 (such as in a database 119 as depicted in FIG. 1). The database 119 may be used for storing information, data etc., for utilization by the computer program 106 (as needed). The sense stimulation signals (also called virtual-reality information) may be predetermined information or may be generated by the processor 102 (as needed on-the-fly). In accordance with a preferred embodiment, animation software may be utilized for generating the virtual-reality images (of the virtual medical-imaging system). For instance, an embodiment of the animation module may include ADOBE (TRADEMARK) AFTER EFFECTS (TRADEMARK of ADOBE) software is a digital visual effects, motion graphics, and compositing application developed by ADOBE SYSTEMS (based in the U.S.). The sense-stimulation signals (also called virtual-reality information) are to be experienced by the patient 900 in response to the output transducers 114 (A) receiving the sense-stimulation signals from the processor 102 of the computer system 100, and (B) providing the sense-stimulation signals as sensations to the patient 900 (once the patient 900 is positioned proximate to the output transducers 114, etc.). For instance, the sense-stimulation signals (sensory output signals or sensory-stimulation information or data) may include: (A) visual sensations (images); (B) audio sensations (noises); and/or (C) tactile sensations (vibrations), etc.

Referring to the embodiment as depicted in FIG. 1 and FIG. 3, the computer program 106 is configured to instruct the processor 102 to execute a third operation 306, including providing (transmitting) patient-training information to the patient 900 via at least one of the output transducers 114. The patient-training information may be called patient-instruction signals. The patient-training information may include, for instance, programmed instructions, guidance, sensory feedback, etc., and any equivalent thereof. The patient-instruction signals are configured to train (provide relevant or tailored training information to) the patient 900; this is done in such a way that the patient 900 may improve the state of calmness of the patient 900. More specifically, the patient-training information may include (and is not limited to): (A) audio instructions for suggested breathing patterns, (B) visualization techniques, and/or (C) verbal suggestions, etc. A brain-visualization technique may include a cognitive process of purposefully generating visual mental imagery, with the eyes of the patient 900 opened or closed, simulating or recreating visual perception, in order to maintain, inspect, and transform those images, and thereby modifying the associated emotions or feelings (that may be felt by or imagined by the patient 900); the intent is to provide to the patient 900 an experience of subsequent beneficial physiological, psychological, or social effect, such as expediting the healing of wounds to the body, minimizing physical pain, alleviating psychological pain including anxiety, sadness, and low mood, improving self-esteem or self-confidence, and enhancing the capacity to cope when interacting with the virtual sense information of a medical-imaging system. The patient-training information is configured to suggest to the patient 900 various cognitive strategies (for how to manage themselves) in order to achieve preferred bodily states (predetermined bodily state), such as remaining motionless or calm, while the output collection 112 of the output transducers 114 continues to transmit (convey) the sensory-stimulation information (simulation information) to the patient 900.

Referring to the embodiment as depicted in FIG. 1 and FIG. 3, the computer program 106 is configured to instruct the processor 102 to execute a fourth operation 308, including performing in-situ adjustments (on-the-fly adjustments) to the sensory-stimulation information to be transmitted to the patient 900 via the output transducers 114 (depending on the data collected from the biometric-information sensors 110 from a prior motion performance of the patient 900). It will be appreciated that this operation may include iterative steps or repeated steps (operations) for predetermined duration of time as may be required to assist the training (learning) progress of the patient 900.

Referring to the embodiment as depicted in FIG. 1 and FIG. 3, the computer program 106 is configured to instruct the processor 102 to execute a fifth operation 310, including transmitting the patient-training information to the patient 900 via at least one of the output transducers 114. In this manner, the patient 900 may be able to train (learn) to remain motionless while the patient 900 receives the patient-training information via at least one of the output transducers 114 (preferably for a predetermined duration of time).

Referring to the embodiment as depicted in FIG. 1 and FIG. 3, the computer program 106 is configured to instruct the processor 102 to execute a sixth operation 312, including: (A) transmitting the sensory-stimulation information (such as the virtual images of the medical-imaging system) via the transducers to the patient 900, and (B) transmitting the patient-training information (such as the verbal suggestions for remaining calm) via the output transducers 114 to the patient 900. Preferably, the operations for (A) and (B) are performed over an overlapping time period (or more preferably at the same time). The patient-training information is usable by the patient 900 in such a way that the patient 900 may teach himself or herself (by utilizing the feedback provided by the computer system 100) a predetermined task, such as remaining physically motionless, while at the same time the patient 900 continues to receive (concurrently receives) the sensory-stimulation information provided via the output transducers 114.

Referring to the embodiment as depicted in FIG. 1 and FIG. 3, the computer program 106 is configured to instruct the processor 102 to execute a seventh operation 314, including adapting (changing or altering) the transmission of the sensory-stimulation information, via the output transducers 114 to the patient 900, based on the biometric information (physiological information) received from at least one of the biometric-information sensors 110.

Referring to the embodiment as depicted in FIG. 1 and FIG. 3, the computer program 106 is configured to instruct the processor 102 to execute an eighth operation 316, including: (A) detecting any changes in the physiological information (as provided by the biometric-information sensors 110); and (B) determining whether the patient 900 has remained motionless for a predetermined period of time (based on the information provided by the biometric-information sensors 110); and (C) changing the sensory-stimulation information (to be sent to the patient 900 via the output transducers 114), and (D) transmit the change of the sensory-stimulation information to the patient 900 (via the output transducers 114) for the case where the determination was made that the patient 900 has remained motionless for a predetermined period of time.

Referring to the embodiment as depicted in FIG. 1 and FIG. 3, the computer program 106 is configured to instruct the processor 102 to execute a ninth operation 318, including stopping transmission (terminating the training session) of the sensory-stimulation information to the patient 900 (via all of the output transducers 114) for the case where the computer program 106 determines that the patient 900 has failed to remain motionless for a predetermined period of time (based on the information provided to the processor 102 from the biometric-information sensors 110).

Referring to the embodiment as depicted in FIG. 1 and FIG. 3, the computer program 106 is configured to instruct the processor 102 to execute a tenth operation 320, including (A) adapting (changing or altering) the sensory-stimulation information (to be provided to the patient 900 via the output transducers 114); and (B) transmitting the altered version of the sensory-stimulation information, via at least one of the output transducers 114 to the patient 900, for the case where at least one of the biometric-information sensors 110 provides, to the processor 102, a measurement signal indicating that the patient 900 has remained motionless for a predetermined time period.

Referring to the embodiment as depicted in FIG. 1 and FIG. 3, the computer program 106 is configured to instruct the processor 102 to execute an eleventh operation 322, including computing a score (an objective score indication) indicating a degree of a potential image quality that may be related to a hypothetical medical image generated by a real medical-imaging system (for the case where the training is stopped, and the real medical-imaging system was to be hypothetically used for generating a real medical image of the patient 900 at the current level of training that the patient 900 has received to date).

Referring to the embodiment as depicted in FIG. 1 and FIG. 3, the computer program 106 is configured to instruct the processor 102 to execute a twelfth operation 324, including computing (providing) a quality-computation indication for a potential medical-image quality associated with a hypothetical medical image to be generated by a real medical-imaging system. The quality-computation indication may depend on various factors, such as the data gathered during the training session provided to the patient 900, etc. The quality-computation indication may be a function of the measurement signals (biometric-information signals) received from at least one of the biometric-information sensors 110 (and recorded during the training period) that shows the motion (and/or other) performance measurements of the patient 900 (for training to remain motionless under simulated conditions between the patient 900 and the images of a virtual medical-imaging system).

Referring to the embodiment as depicted in FIG. 1 and FIG. 3, advantageously, the computer system 100 may be utilized before a medical procedure as a screening tool. The computer program 106 is configured to compute (determine) potential outcomes, such as: (A) whether additional training (from the computer system 100) may be required by the patient 900; or (B) whether the patient 900 may require sedation during the medical procedure (in view of the accumulated training provided by the computer system 100 to the patient 900 to date and in view of the improvement made by the patient 900 during training); or (C) whether the patient 900 may be ready to experience the real medical-imaging system with an improved confidence (an adequate degree of confidence) in the potential image quality resulting from the real medical image that may be generated by the real medical-imaging system (in view of the accumulated training provided by the computer system 100 to the patient 900 to date). The computer system 100 may improve the outcome for an acceptable medical image to be generated by the real medical-imaging system (such as, diagnostic imaging quality of pictures, surgery); preferably, this is done without sedation (or less sedation) for the patient 900.

Referring to the embodiment as depicted in FIG. 1 and FIG. 3, the computer program 106 is configured to instruct the processor 102 to execute a thirteenth operation 326, including recording the measurement data (provided by the biometric-information sensors 110) indicating the motions of the patient 900 (to the memory device 104).

Referring to the embodiments as depicted in FIG. 1 and FIG. 3, the computer program 106 is configured to instruct the processor 102 to execute a fourteenth operation 328, including computing (based on the data that was received from the output transducers 114 and then recorded to memory) the improvements (in the reduction of physical motions) made by the patient 900 as a result of the patient 900 attempting to remain motionless while: (A) the virtual stimulation (of sense stimulation signals) is presented to the patient 900 via at least one of the output transducers 114; and (B) the biometric information (the physiological measurements) of the patient 900 is collected via at least one of the biometric-information sensors 110.

Referring to the embodiment as depicted in FIG. 1 and FIG. 3, the computer program 106 is configured to instruct the processor 102 to execute a fifteenth operation 330, including computing (and then providing) an estimation that indicates an amount of additional stimulation-training time that may be needed by the patient 900 (based on a set of predetermined rules or criteria), in order to improve the potential medical-image quality that may be acceptable (for the case where the real medical-imaging system was to be hypothetically utilized to make or generate a medical image of the patient 900).

Referring to the embodiment as depicted in FIG. 1 and FIG. 3, the computer program 106 is configured to instruct the processor 102 to execute a sixteenth operation 332, including providing (transmitting) an interactive game, via the output transducers 114 to the patient 900. The interactive game may include presentation of an image of an avatar (to the patient 900), and the avatar is controllable by the motions made by the patient 900 (as determined from the biometric-information sensors 110) while the patient 900 undergoes the training process. An avatar is an icon or figure representing a particular personality or entity in a video game, etc. For instance, the image of the avatar may include a happy avatar for the case where the patient 900 remains motionless (as determined over a predetermined period of time based on the information provided by the biometric-information sensors 110). The image of the avatar may include a sad avatar for the case where the patient 900 does not remain motionless (as determined over a predetermined period of time based on the information provided by the biometric-information sensors 110).

Referring to the embodiment as depicted in FIG. 1 and FIG. 3, the computer program 106 is configured to instruct the processor 102 to execute a seventeenth operation 334, including providing (executing) a virtual-reality game (to the patient 900). The game (virtual game) includes providing the movements of an avatar while providing the virtual stimulation of a virtual medical-imaging system and/or a real medical-imaging system (for presentation to the patient 900), etc.

FIG. 4 depicts a schematic view (representation) of an embodiment of a flow chart (control logic) to be utilized by the first software module 120 of the computer program 106, which is to be executed by the processor 102 of the computer system 100 of FIG. 1.

Referring to the embodiment as depicted in FIG. 4, the first software module 120 is configured to provide control logic (similar to the logic for the computer program 106) for controlling the components associated with the computer system 100. The first software module 120 is configured to instruct the processor 102 to execute a providing operation 402, including instructing the processor 102 to provide (execute) a virtual-reality stimulation of a virtual medical-imaging system, via the output transducers 114, to the patient 900. For instance, the first software module 120 is configured to transmit a stimulation (simulation) configured to stimulate the senses (via sight, hearing, etc.) of the patient 900 as to what the patient 900 might sense (feel, see) for the case where the patient 900 might be positioned in or near a medical-imaging system (whether real or virtual). The providing operation 402 may include instructing the processor 102 to provide visual images to the patient 900, via the output transducers 114, sound recordings, audio recordings and/or haptic sensations of the real medical-imaging system. The visual images of the real medical-imaging system represent a virtual medical-imaging system. The visual images of the real medical-imaging system may include: (A) an image of a room (preferably with a suitable decoration design for viewing by the patient 900); (B) an image representing a three-dimensional model of a medical-imaging system, such as including a chair (or a bed, etc.) for the patient 900; and (C) a visual image representing a technician that may be seen beyond a window (positioned in a control room), etc. The first software module 120 is configured to urge the processor 102 to execute movement operations 404 (via the output transducers 114), including: (A) providing an animation of the movement of a virtual patient-support platform (such as, a virtual bed) relative to a virtual image of a medical-imaging system; (B) playing the virtual audio information associated with a virtual medical-imaging system (such as playing the virtual audio information for a duration in response to receiving a suitable command from the technician or operator via the user interface); (C) ending the playing of the virtual audio information (in response to receiving a stop request of the operator via the user interface); and (D) playing an animation for showing the movement of the virtual patient-support platform move away from the virtual image of the medical-imaging system, etc.

Referring to the embodiment as depicted in FIG. 4, the first software module 120 is configured to urge the processor 102 to execute a recording operation 406, including instructing the processor 102 to record, to the database 119 (as depicted in FIG. 1), patient metrics (such as, the measured movements of the patient 900 as detected by at least some of the biometric-information sensors 110) throughout (during at least a part of) the stimulation (simulation) of the virtual medical-imaging system. The measurement data (provided by the sensors and to be recorded) may include: (A) a timestamp, (B) the absolute head position and rotation of the patient 900, (C) the position change vector from the last measurement, (D) the rotation change vector from the last measurement, (E) the decibel level (provided by the microphone), (F) the heart rate of the patient 900, (G) the blood pressure of the patient 900, (H) the blood oxygen level of the patient 900, and/or (I) the temperature of the patient 900, etc. Preferably, the first software module 120 is configured to record the data on a frequent basis (such as, every second, etc.).

FIG. 5 depicts a schematic view (representation) of an embodiment of a flow chart (control logic) to be utilized by the second software module 122 of the computer program 106 to be executed by the processor 102 (utilized by the computer system 100 of FIG. 1).

Referring to the embodiment as depicted in FIG. 5, the second software module 122 is configured to provide control logic (similar to the logic for the computer program 106) for controlling the components associated with the computer system 100.

The second software module 122 includes a first game-delivery operation 502A configured to instruct the processor 102 to provide (execute, transmit) a game (via the biometric-information sensors 110 and/or the output transducers 114, etc.) to the patient 900. The game is configured to interactively train the patient 900 to remain motionless during at least a predetermined period of time during the game (such as, a stay-still game), such as for when the patient 900 is positioned in a virtual medical-imaging system. Preferably, the second software module 122 is usable with children, who may (preferably) be main target demographics.

Referring to the embodiment as depicted in FIG. 5, the second software module 122 includes a second game-delivery operation 502B configured to instruct the processor 102 to wait while the patient 900 is lying on the real bed in a preferred orientation (such as, looking upwardly). The patient 900 wears at least one of the output transducers 114 (such as, the virtual-reality headset 200) while the patient 900 lies in the real bed 202 (as depicted in FIG. 2).

Referring to the embodiment as depicted in FIG. 5, the second software module 122 includes a third game-delivery operation 502C configured to instruct the processor 102 to teach (via the biometric-information sensors 110 and/or the output transducers 114, etc.) the patient 900 not to move their head (that is, to remain motionless) for a predetermined period of time (that is, to train the patient 900 to remain as still as possible for the duration of time allotted).

Referring to the embodiment as depicted in FIG. 5, the second software module 122 includes a fourth game-delivery operation 502D configured to instruct the processor 102 to (A) determine (based on the data received from at least one of the biometric-information sensors 110) that there is a positive outcome for the patient 900 (such as, awarding the patient 900 for the case where the patient 900 has managed to remain relatively still or motionless, along with audio and visual cues or awards), and (B) provide an indication of a reward for the patient 900 (via the output transducers 114) as positive reinforcement for the patient 900.

Referring to the embodiment as depicted in FIG. 5, the second software module 122 includes a fifth game-delivery operation 502E configured to instruct the processor 102 to provide (via the output transducers 114 to the patient 900) a notification that the patient 900 has failed to remain relatively motionless (for the case where at least one of the biometric-information sensors 110 has provided to the processor 102 an indication that the patient 900 has moved, and the processor 102 has determined that the patient movement has occurred during a predetermined period of time).

Referring to the embodiment as depicted in FIG. 5, the second software module 122 includes a sixth game-delivery operation 502F configured to instruct the processor 102 to (A) collect the data received from at least one of the biometric-information sensors 110 throughout the time the game is executing (that is, delivered to the patient 900), and (B) record the data received from at least one of the output transducers 114 (such as, about the head position and audio levels, etc.).

Referring to the embodiment as depicted in FIG. 5, the second software module 122 includes a seventh game-delivery operation 502G configured to instruct the processor 102 to provide to the patient 900, via the output transducers 114, the game for a predetermined period of time, such as for about five minutes, ten minutes, 20 minutes, etc.

The second software module 122 includes an eighth game-delivery operation 502H configured to instruct the processor 102 to provide, via at least one of the output transducers 114 to the patient (such as a patient-output display or the virtual-reality headset 200), an image of an avatar (animated avatar). For instance, the avatar may appear to be flying (elevated) above the patient 900 (once the patient 900 is wearing the patient output display or the virtual-reality headset 200).

Referring to the embodiment as depicted in FIG. 5, the second software module 122 includes a ninth game-delivery operation 502I configured to instruct the processor 102 to provide, via at least one of the output transducers 114 to the patient 900, an image of the avatar along with a visual indicator (such as, a container or a battery, etc.) that shows the progress (degree of improvement) to the patient 900.

Referring to the embodiment as depicted in FIG. 5, the second software module 122 includes a tenth game-delivery operation 502J configured to instruct the processor 102 to provide, via at least one of the output transducers 114 to the patient 900, a visual indicator positioned at a region on the patient display device or the virtual-reality headset 200 (for instance, right in front of the patient 900) so that the patient 900 does not need to move his head to look around for the visual indicator (information).

Referring to the embodiment as depicted in FIG. 5, the second software module 122 includes an eleventh game-delivery operation 502K configured to instruct the processor 102 to provide, via at least one of the output transducers 114 to the patient 900, the visual indicator that appears to be “filling up” while the patient 900 manages to remain still or motionless (as determined by the processor 102 based on the information provided by the biometric-information sensors 110 over a predetermined period of time; that is, no head movement was detected for the patient 900 for at least three minutes, etc.).

Referring to the embodiment as depicted in FIG. 5, the second software module 122 includes a twelfth game-delivery operation 502L configured to instruct the processor 102 to provide the visual indicator, via at least one of the output transducers 114 to the patient 900, that indicates an emptying state for the visual indicator for the case where the processor 102 determines that the patient 900 has physically moved (as detected by at least one of the biometric-information sensors 110).

Referring to the embodiment as depicted in FIG. 5, the second software module 122 includes a thirteenth game-delivery operation 502M configured to instruct the processor 102 to provide the visual indicator, to the patient 900 via at least one of the output transducers 114, as being slowly filled up for the case where the patient 900 manages to remain physically still (as detected by at least one of the biometric-information sensors 110 over a predetermined period of time).

Referring to the embodiment as depicted in FIG. 5, the second software module 122 includes a fourteenth game-delivery operation 502N configured to instruct the processor 102 to stop transmission of the game to the patient 900 (via at least one of the output transducers 114) once the visual indicator is shown as being filled (to capacity).

Referring to the embodiment as depicted in FIG. 5, the second software module 122 includes a fifteenth game-delivery operation 502O configured to instruct the processor 102 to provide via at least one of the output transducers 114 to the patient 900 (A) the visual image of the avatar in a smiling state (a happy state) for the case where at least one of the biometric-information sensors 110 has indicated that the patient 900 has managed to remain still for a predetermined period of time, and (B) a feedback signal, such as from the avatar, that the patient 900 is performing well at staying still (a form of a reward or patient encouragement).

Referring to the embodiment as depicted in FIG. 5, the second software module 122 includes a sixteenth game-delivery operation 502P configured to instruct the processor 102 to provide to the patient 900, via at least one of the output transducers 114, (A) an image of the avatar in a frown state (a sad state) for the case where at least one of the biometric-information sensors 110 has indicated that the patient 900 has remained in motion (failed to remain motionless) as detected by at least one of the biometric-information sensors 110, and (B) a visual clue (such as from the avatar) that the patient 900 is performing badly at staying still or motionless) and/or (C) an audio indication that suggests that the patient 900 may try to move less, or be still (and that, for instance, the avatar may smile for the patient 900 as patient encouragement or a feel-good moment).

Referring to the embodiment as depicted in FIG. 5, the second software module 122 includes a seventeenth game-delivery operation 502Q configured to instruct the processor 102 to provide, via at least one of the output transducers 114 such as the virtual-reality headset 200, the image of the avatar to the patient 900. The image of the avatar plays (animates) a list of opening narrations (an avatar audio file), and performs animations (an avatar image-movement file) once the patient 900 is wearing the virtual reality headset, etc.

Referring to the embodiment as depicted in FIG. 5, the second software module 122 includes an eighteenth game-delivery operation 502R configured to instruct the processor 102 to provide, via at least one of the output transducers 114, (A) a progress indicator to the patient 900, and (B) an indication of filling of the progress indicator (for indicating the duration of time that the patient 900 has managed to remain motionless based on the information received by the processor 102 from at least one of the biometric-information sensors 110, etc.).

Referring to the embodiment as depicted in FIG. 5, the second software module 122 includes a nineteenth game-delivery operation 502S configured to instruct the processor 102 to provide, via at least one of the output transducers 114, the image of the avatar to the patient 900. The avatar may be depicted as saying something to the patient 900, preferably randomly, from a BAD narration list for the case where the patient 900 physically moves (the patient 900 cannot remain motionless) as detected by at least one of the biometric-information sensors 110 for a predetermined period of time.

Referring to the embodiment as depicted in FIG. 5, the second software module 122 includes a twentieth game-delivery operation 502T configured to instruct the processor 102 to provide, via at least one of the output transducers 114, the image of the avatar to the patient 900. The avatar may be depicted as saying (speaking) something, preferably randomly, from the GOOD narration list (A) for the case where the patient 900 manages to remain physically still or motionless (based on the information collected from at least one of the biometric-information sensors 110), or (B) for the case where the patient 900 remains still (for a predetermined duration of time that may be randomly selected) based on the information provided by at least one of the biometric-information sensors 110 to the processor 102.

Referring to the embodiment as depicted in FIG. 5, the second software module 122 includes a twenty-first game-delivery operation 502U configured to instruct the processor 102 to provide, via at least one of the output transducers 114, the image of the avatar to the patient 900. The avatar may be depicted as remaining idle and not saying anything (for example, the avatar includes a fairy flapping wings, etc.).

Referring to the embodiment as depicted in FIG. 5, the second software module 122 includes a twenty-second game-delivery operation 502V configured to instruct the processor 102 to provide, via at least one of the output transducers 114, the image of the avatar to the patient 900 along with an ending narration and corresponding animation.

Referring to the embodiment as depicted in FIG. 5, the second software module 122 includes a twenty-third game-delivery operation 502W configured to instruct the processor 102 to record the metrics (information) received from at least one of the biometric-information sensors 110 and/or at least one of the output transducers 114 (similar to the first software module 120).

Referring to the embodiment as depicted in FIG. 5, the second software module 122 includes a twenty-fourth game-delivery operation 502X configured to instruct the processor 102 to collect (receive or process) prior imaging details (where available) for all patients (the historical data collected for the patient population) by the operator (the study investigators). The prior imaging details may include (and are not limited to): (A) a type of examination (modality, with or without contrast, etc.); (B) a date; and (C) an imaging conclusion (judgement of the medical image). It will be appreciated that imaging quality for prior medical images (such as an MRI scan) may be assessed where applicable by a blinded third-party medical specialist (such as a radiologist), and may be ranked (for instance, by using a score from one to four (1-4), for instance with two positive points for MRI signal adequacy and resolution, and two positive points for lack of motion artifact and lack of repeated sequences). The metrics may include (A) a heart rate; (B) a motion indication; (C) a breathing indication; and (D) a GAD-7 questionnaire. The metrics may be collected and inputted to a prediction module 127 (also called a prediction algorithm or an artificial-intelligence algorithm, etc.). The prediction algorithm is configured to create deep learning for computing outcomes for image quality. The GAD-7 questionnaire is useful in primary care and mental health settings as a screening tool and symptom severity measure for the four most common anxiety disorders (Generalized Anxiety Disorder, Panic Disorder, Social Phobia and Post Traumatic Stress Disorder).

Referring to the embodiment as depicted in FIG. 5, the second software module 122 includes a twenty-fifth game-delivery operation 502Y configured to instruct the processor 102 to (A) compare the data of the patient 900 to a robust database of prior similar metrics, and (B) predict image quality of a hypothetical medical image to be generated by a real medical-imaging system. For instance, a number is associated with the image quality, such as numbers (I) to (IV). The numbers (I), (II), (III) and (IV) refer to a grading number for the image quality: the number (I) represents the poorest image quality, and the number (IV) represents the best image quality. Reference is made to the following publication for image quality: CT ANGIOGRAPHY OF THE HEAD AND NECK: IMAGE QUALITY AS A FUNCTION OF PHYSIOLOGIC PARAMETERS, A TOMOGRAPHY-ULTRASOUND FUSION INVESTIGATION; authors: H. Maresky, et al.; Radiologic Association Annual Meeting, Israel; date of publication: Oct. 28-30, 2015.

Referring to the embodiment as depicted in FIG. 5, the second software module 122 includes a twenty-sixth game-delivery operation 502Z configured to instruct the processor 102 to provide (via the display device 118 depicted in FIG. 1) an indication of a proposed training program (based upon, or computed from, the execution of a prediction algorithm). The prediction algorithm (software) may be utilized for developing the training program to be used with the patient 900. For instance, the prediction algorithm may be applied to multiple prior patients (with similar metrics). The prediction algorithm is configured to identify the best outcome for image quality for a particular patient. For instance, the prediction algorithm may be utilized for predicting the image quality (such as, a score of 2.5 points out of 4 possible points, etc.). For instance, the second software module 122 is configured to provide an indication of a training program of a quantity of two 20-minute long simulator sessions (training sessions), or a quantity of three 5-minute long simulator sessions (training sessions), etc. The prediction algorithm (also called predictive analysis software) may include an artificial intelligence biofeedback algorithm that uses resonance of cardiovascular system for strengthening the baroreflex (the modulation of autonomic nervous system of the patient 900) for the control of emotional reactivity of the patient 900. The prediction algorithm may include any algorithm for machine learning. The prediction algorithm (modelling) uses statistics to predict outcomes. The prediction algorithm may be chosen on the basis of detection theory to try to guess the probability of an outcome given a set amount of input data. The prediction algorithm may use one or more classifiers in trying to determine the probability of a set of data belonging to another set. The prediction algorithm may be synonymous with, or largely overlapping with, the field of machine learning, as it is more commonly referred to in academic or research and development contexts. The prediction algorithm (the prediction module 127 as depicted in FIG. 1) may also be called predictive analytics. For instance, embodiments of the prediction algorithm may include the MICROSOFT (TRADEMARK) AZURE MACHINE LEARNING STUDIO (TRADEMARK of MICROSOFT) prediction algorithm (MICROSOFT is based in the U.S.), the ORACLE (TRADEMARK) CRYSTAL BALL (TRADEMARK of ORACLE) prediction algorithm (ORACLE is based in the U.S.) and/or the IBM (TRADEMARK) SPSS PREDICTIVE ANALYTICS ENTERPRISE (TRADEMARK of IBM) prediction algorithm (IBM is based in the U.S.).

Referring to the embodiment as depicted in FIG. 1, the prediction module 127 (also called an Artificial Intelligence program), or AI program, may be configured to enable the computer system to learn from experience and perform human-like tasks. The prediction module 127 provides the computer system the ability to learn in an intelligent way. The prediction module 127 may be configured for processing vast amounts of data generated on a periodic basis. By strategically applying (using) the prediction module 127 to certain processes, insight gathering and task automation occur at an otherwise unimaginable rate and scale. Parsing through the mountains of data created by humans, the prediction module 127 may perform intelligent searches, interpreting both text and images to discover patterns in complex data, and then act on those learnings. The prediction module 127 may be configured to enable the computer system to understand the meaning of human language, learn from experience, and make predictions, respectively.

Referring to the embodiment as depicted in FIG. 1, for instance, machine learning, or ML, is an application of the prediction module 127 that provides the computer system with the ability to automatically learn and improve from experience without being explicitly programmed. ML focuses on the development of algorithms that can analyze data and make predictions. There are many ways to implement the prediction module 127.

Referring to the embodiment as depicted in FIG. 1, for instance, deep learning is a subset of machine learning (ML) that employs artificial neural networks that learn by processing data. Artificial neural networks mimic the biological neural networks in the human brain. Multiple layers of artificial neural networks work together to determine a single output from many inputs, for example, identifying the image of a face from a mosaic of tiles. The computer system learns through positive and negative reinforcement of the tasks they carry out, which requires constant processing and reinforcement to progress. For instance, another form of deep learning is speech recognition, which enables the voice assistant in phones to understand questions like, “Hey Siri, How does artificial intelligence work?” Neural networks enable deep learning. Neural networks are computer systems modeled after neural connections in the human brain. The artificial equivalent of a human neuron is a perceptron. Just like bundles of neurons create neural networks in the brain, stacks of perceptrons create artificial neural networks in computer systems. Neural networks learn by processing training examples. This process analyzes data many times to find associations and give meaning to previously undefined data. Through different learning models, like positive reinforcement, the computer system taught it has successfully identified the object. For instance, cognitive computing may be another essential component of the prediction module 127. Cognitive computing seeks to recreate the human thought process in a computer model, in this case, by understanding human language and the meaning of images. Together, cognitive computing and artificial intelligence strive to endow the computer system with human-like behaviors and information processing abilities. For instance, Natural Language Processing software (NLP software), allows computers to interpret, recognize, and produce human language and speech. The NLP software may enable seamless interaction with the computer system by teaching the computer system to understand human language in context and produce logical responses. For instance, computer vision is a technique that implements deep learning and pattern identification to interpret the content of an image; including the graphs, tables, and pictures within PDF documents, as well as, other text and video. Computer vision may be included in the prediction module 127 for enabling the computer system to identify, process and interpret visual data.

Referring to the embodiment as depicted in FIG. 5, the second software module 122 includes a twenty-seventh game-delivery operation 502AA configured to instruct the processor 102 to provide (via the display device 118) an assessment indication of an assessment of a predicted image quality of a hypothetical medical image to be generated by a real medical-imaging system. The assessment indication may (would) track the patient's change in the expected image quality score (for instance, a point value of 2.5, 3.0, 3.5 points, etc.). The operator may use the prediction algorithm as needed (on an ongoing basis) until the computer image quality has reached at least a desired score (for instance, 3.5 points or higher). This computation helps to ensure where or when the patient 900 does use the real medical-imaging system, the real medical-imaging system may then be in a better position to provide (generate) a better quality diagnostic medical image (preferably of the highest possible image quality) that may be generated from the real medical-imaging system. It may be appreciated that it is important to reduce motion artifact in a medical image to be captured by the real medical-imaging system (especially for the pediatric population). This may assist in proper diagnosis of the medical condition of the patient 900. Reference is made to the following publication: PROSPECTIVE MOTION CORRECTION IMPROVES DIAGNOSTIC UTILITY OF PEDIATRIC MRI SCANS. Pediatric radiology. 1 Dec. 2011; 41(12):1578-82 that was authored by Kuperman J. M., et al. For diagnostic purposes, the image quality of a medical image may be important in order to arrive at a correct diagnosis of a potential disease process, and to rule out sinister disease processes.

Referring to the embodiment as depicted in FIG. 5, the second software module 122 includes a twenty-eighth game-delivery operation 502BB configured to instruct the processor 102 to compute and then provide (via the display device 118) an indication of a potential medical image quality. The medical image quality that is computed may indicate, potentially, how good (or bad) the medical image may turn out if the medical-imaging system was to hypothetically generate a real medical image of the patient 900 (based on the training provided to the patient 900 and based on the progress made by the patient 900 to date for learning to remain motionless). The computation may be based on the historical training provided to date along with the collected data provided by the biometric-information sensors 110.

Referring to the embodiment as depicted in FIG. 5, the second software module 122 includes a twenty-ninth game-delivery operation 502CC configured to instruct the processor 102 to utilize a biofeedback operation including: (A) monitoring biometric information (such as, the heart rate) of the patient 900 (via at least one of the biometric-information sensors 110), and (B) feeding back information (regarding the biometric information or measurements) to the patient 900 (via at least one of the output transducers 114) so that the patient 900 may adjust his responses to the stimulus (sensation signals), etc.

Referring to the embodiment as depicted in FIG. 5, the second software module 122 includes a thirtieth game-delivery operation 502DD configured to instruct the processor 102 to provide (via at least one of the output transducers 114) a progress indication to the patient 900 (by way of a chat box), and the progress indication may say (A) GOOD for staying still (remaining motionless), or (B) STAY STILL for when the patient 900 is detected in motion by at least one of the biometric-information sensors 110. It will be appreciated that the physiologic parameters of the patient 900 may improve the imaging quality of a real medical image (to be generated by the real medical-imaging system) by utilization of biofeedback principles (i.e.: training of the brain stem of the patient 900, which modulates heart rate, breathing rate and fear, for training of the amygdala, etc.).

Referring to the embodiment as depicted in FIG. 5, the second software module 122 includes a thirty-first game-delivery operation 502EE configured to instruct the processor 102 to collect physiologic parameters or biometric signals of the patient 900 (such as, an electrocardiogram (ECG), heart rate (HR), a breathing pattern, etc.) from at least one of the biometric-information sensors 110 (depicted in FIG. 1) once the sensors are accordingly attached to (coupled to) the patient 900.

FIG. 6 depicts a schematic view (representation) of an embodiment of a flow chart (control logic) to be utilized by the third software module 124 of the computer program 106 to be executed by the processor 102 (to be utilized by the computer system 100) of FIG. 1.

Referring to the embodiment as depicted in FIG. 6, the third software module 124 is configured to instruct the processor 102 to provide (via at least one of the output transducers 114) anxiety-relief training (such as, sensory signals having patient instructions) to the patient 900 (as depicted in FIG. 2). This is done in such a way that the patient 900 may experience relative calm (and/or relatively motionless behavior) while the virtual medical-imaging system is made to appear operational (via sense signals sent to the patient 900) such as when the patient 900 is positioned in the medical-imaging system, etc. Preferably, the third software module 124 is used with children (a preferred target demographic). Preferably, the third software module 124 is for use with the patient 900 lying in a real bed 202 and while the patient 900 looks upwardly while wearing the virtual-reality headset 200 (as depicted in FIG. 2).

Referring to the embodiment as depicted in FIG. 6, the third software module 124 includes a first anxiety-relief training operation 602A, including instructing the processor 102 to transmit information (via at least one of the output transducers 114). The information keeps (helps to maintain) the attention of the patient 900 in an occupied and focused state based on the information provided to the patient 900 (preferably, while reducing or blocking out (ignoring, at least in part) any other unwanted stimulation that may (could) interfere with the attention of the patient 900 during a training session).

Referring to the embodiment as depicted in FIG. 6, the third software module 124 includes a second anxiety-relief training operation 602B, including instructing the processor 102 to provide (via at least one of the output transducers 114) an image of an animated avatar to be viewed by the patient 900; for instance, the avatar may be seen flying above the patient 900 (for instance, while the patient 900 wears the virtual-reality headset 200).

Referring to the embodiment as depicted in FIG. 6, the third software module 124 includes a third anxiety-relief training operation 602C, including instructing the processor 102 to provide (via the output transducers 114) a visual representation of an environment to the patient 900. For instance, the visual representation may include a colorful underwater scene with plants floating and fish swimming around (or other relaxing environments).

Referring to the embodiment as depicted in FIG. 6, the third software module 124 includes a fourth anxiety-relief training operation 602D, including instructing the processor 102 to provide, via at least one of the output transducers 114 (such as, the virtual-reality headset 200), an image of the avatar that visually appears, and the image of the avatar is made to recite (say) or play a list of opening narrations (audio signals) and/or animations (visual movements) to the patient 900.

Referring to the embodiment as depicted in FIG. 6, the third software module 124 includes a fifth anxiety-relief training operation 602E, including instructing the processor 102 to (A) provide, via at least one of the output transducers 114, a visual representation of the avatar to the patient 900, and (B) provide the avatar reciting (saying), via at least one of the output transducers 114, something randomly selected from a BAD narration list for the case where the patient 900 is detected as having moved or has failed to remain motionless for a predetermined period of time (the motion of the patient 900 is detected by the biometric-information sensors 110 and appropriate motion-indicating signals are accordingly transmitted to the processor 102).

Referring to the embodiment as depicted in FIG. 6, the third software module 124 includes a sixth anxiety-relief training operation 602F, including instructing the processor 102 to (A) provide, via at least one of the output transducers 114, a visual representation of the avatar (to the patient 900), and (B) provide the avatar reciting (saying) something randomly (to the patient 900), such as a GOOD narration list and/or playing animations for the case where the patient 900 manages to remain motionless or still for a predetermined duration of time (this time duration may be randomly chosen by the operator if desired). The motions of the patient 900 are provided to the processor 102 as detected by the biometric-information sensors 110 (so that the processor 102 may compute whether the patient 900 has remained motionless for a period of time).

Referring to the embodiment as depicted in FIG. 6, the third software module 124 includes a seventh anxiety-relief training operation 602G, including instructing the processor 102 to (A) provide, via at least one of the output transducers 114, a visual representation of the avatar (to the patient 900) opening a movie screen, and (B) provide, via at least one of the output transducers 114, an animated movie to the patient 900, and (C) remove the screen, as presented via at least one of the output transducers 114, once the animated movie is completed (ended), so that the avatar may resume animation to the patient 900 (via at least one of the output transducers 114).

Referring to the embodiment as depicted in FIG. 6, the third software module 124 includes an eighth anxiety-relief training operation 602H, including instructing the processor 102 to provide, via at least one of the output transducers 114, a visual representation of the avatar (to the patient 900), while the avatar is shown as being idle and not saying anything. Preferably, the avatar may be depicted as being in a subtle idle animation (for example, the avatar includes a fairy slowly flapping her wings and simply waiting).

Referring to the embodiment as depicted in FIG. 6, the third software module 124 includes a ninth anxiety-relief training operation 602I, including instructing the processor 102 to provide, via at least one of the output transducers 114, a visual representation of the avatar (to the patient 900). The image of the avatar is shown playing (animating) a list of ending narrations and/or animations to the patient 900 such as for the case where the time has expired (or the operator decides to end the training session), etc.

FIG. 7 depicts a schematic view (representation) of an embodiment of a flow chart (also called the control logic) to be utilized by a calibration software module 126 (a software module) of the computer program 106 to be executed by the processor 102 of the computer system 100 of FIG. 1.

Referring to the embodiment as depicted in FIG. 7, the calibration software module 126 is configured to instruct the processor 102 to provide a calibration operation 702 for calibrating the computer system 100 and the components of the computer system 100. The calibration operation 702 includes providing an operator-interface screen on the display device 118 to an operator (a technician) as depicted in FIG. 2.

Referring to the embodiment as depicted in FIG. 7, the calibration operation 702 includes a first calibration operation 702A, including instructing the processor 102 to electronically interact with a virtual-reality headset 200 (depicted in FIG. 2, which is an example of at least one of the output transducers 114 of FIG. 1) to be worn by the patient 900 (as depicted in FIG. 2). The virtual-reality headset 200 is operatively connected to the computer system 100 (via a suitable interface) by interfaces that are generally known to persons of skill in the art.

Referring to the embodiment as depicted in FIG. 7, the calibration operation 702 includes a second calibration operation 702B, including instructing the processor 102 to provide (transmit) an image of a virtual bed to the virtual-reality headset 200. It will be appreciated that the virtual-reality headset 200 displays the virtual bed to the patient 900 (once the patient 900 wears the virtual-reality headset). The virtual bed (generally called the virtual patient-support system) corresponds to, and is calibrated to, a real bed 202 (as depicted in FIG. 2). The patient 900 rests on (horizontally contacts) the real bed, as depicted in FIG. 2. The real bed is generally called a real patient-support system.

Referring to the embodiment as depicted in FIG. 7, the calibration operation 702 includes a third calibration operation 702C, including instructing the processor 102 to wait for the operator to place the virtual-reality headset 200 on a part of (such as, the head of) the real bed 202. For instance, this is done in such a way that the virtual-reality headset 200 is looking down (pointed along) a length of the real bed 202, etc., as may be required.

Referring to the embodiment as depicted in FIG. 7, the calibration operation 702 includes a fourth calibration operation 702D, including instructing the processor 102 to (A) wait to receive a start signal, from the user interface 115, to start the calibration process (such as, for when the operator presses a calibrate field located on the display device 118), and (B) match the position, rotation and height of the virtual bed (the virtual patient support image) with the real bed (the real patient-support surface) as viewed through the virtual-reality headset 200 (as viewed by the patient 900).

Referring to the embodiment as depicted in FIG. 7, the calibration operation 702 includes a fifth calibration operation 702E, including instructing the processor 102 to receive an indication (via the user interface 115) from the operator. The indication may identify any one of the first software module 120, the second software module 122, or the third software module 124 for execution by the processor 102. Preferably, each software module is operated for a corresponding determined (predetermined) time period.

Referring to the embodiment as depicted in FIG. 7, the calibration operation 702 includes a sixth calibration operation 702F, including instructing the processor 102 to receive a start module indication signal, via the user interface 115 (such as, an input device 117 and/or the display device 118 both depicted in FIG. 1). For instance, the start module indication signal indicates that the operator (the technician) has pressed a “start” button on the display device 118 (to start a selected one of the first software module, the second software module or the third software module, etc.).

Referring to the embodiment as depicted in FIG. 7, the calibration operation 702 includes a seventh calibration operation 702G, including instructing the processor 102 to receive, via the user interface, patient information (via the user interface, such as the input device 117) from the operator; this may be performed for the case where a new patient arrives and is ready for receiving training from the computer system 100. The patient information may include (and is not limited to): (A) the name of the patient; (B) the date of birth of the patient; (C) an identification indication for the patient; (D) any previous imaging information and corresponding date (text, single line), (E) the height of the patient, (F) the weight of the patient 900, (G) any developmental delay associated with the patient 900 (information may be limited to a Yes/No response from the operator), and/or (H) the patient type, such as an oncologic-type patient (information may be limited to a Yes/No response from the operator), etc.

Referring to the embodiment as depicted in FIG. 7, the calibration operation 702 includes an eighth calibration operation 702H, including instructing the processor 102 (as depicted in FIG. 1) to receive, via the user interface (that is, the input device 117, such as a mouse, keyboard, etc.), an option indication (signal) from the operator (the technician). Preferably, the option indication (signal) is selected (for instance) from three options (or more options): option (1), option (2) or option (3). Option (1) includes instructing the processor 102 to wait until the allotted time passes (to complete execution of a selected software module). Option (2) includes instructing the processor 102 to perform a graceful stop (that is, to gracefully stop the computer program 106, and the virtual bed may slide out or away from the virtual medical-imaging system, or the game avatar may say goodbye to the patient 900, etc.). Option (3) includes instructing the processor 102 to execute an emergency stop (this option blacks out all visual and audio outputs to the patient 900, and may be used for the case where the patient 900 experiences a panic attack and/or physical discomfort, etc.).

### Concluding Remarks

In view of the foregoing, there is provided an apparatus, comprising: a computer system 100 (a system) including an interface unit 128 being configured to: (I) interface and couple with output transducers 114 configured to receive, and provide to, a patient 900 (A) sensory output signals associated with a virtual medical-imaging system, and (B) patient-training information configured to urge the patient 900 to improve of a state of calmness (in mind and/or body) of the patient 900; and (II) interface and couple with biometric-information sensors 110 configured to generate biometric-information signals in response to a biometric reaction of the patient 900; and (III) transmit, to the patient 900 via the output transducers 114, the sensory output signals associated with the virtual medical-imaging system; and (IV) receive, from the patient 900 via the biometric-information sensors 110, at least one of the biometric-information signals of the patient 900 that represent a biometric-reaction of the patient 900 in response to the patient 900 receiving, via the output transducers 114, the sensory output signals associated with the virtual medical-imaging system; and (V) transmit, to the patient 900 via the output transducers 114, the patient-training information configured to urge the patient 900 to improve the state of calmness of the patient 900. At least some of the patient-training information takes into account at least one of the biometric-information signals that were received via the biometric-information sensors 110.

In view of the foregoing, there is provided a method for providing patient-training information to a patient 900. The method includes (comprises): operation (A) transmitting, to the patient 900 via output transducers 114, sensory output signals associated with a virtual medical-imaging system; and operation (B) receiving, from the patient 900 via biometric-information sensors 110, biometric-information signals of the patient 900 that represent a biometric reaction of the patient 900 in response to the patient 900 receiving, via the output transducers 114, the sensory output signals associated with the virtual medical-imaging system; and operation (C) transmitting, to the patient 900 via the output transducers 114, patient-training information configured to urge the patient 900 to improve a state of calmness of the patient 900. At least some of the patient-training information takes into account at least one of the biometric-information signals that were received via the biometric-information sensors 110.

In view of the foregoing, there is provided an apparatus configured to operate in accordance with the method described the paragraph immediately above.

In view of the foregoing, there is provided a memory device 104 tangibly embodying a computer program 106 configured to be readable by, and executable by, a processor 102 of a computer system 100 (a system), and the computer program 106 configured to operate in accordance with the method described the paragraph immediately above.

In view of the foregoing, there is provided a computer system 100 (a system), comprising: (I) an interface unit 128 being configured to electrically interface and couple with biometric-information sensors 110 configured to receive biometric-information signals from a patient 900 in response to a biometric reaction of the patient 900; and the interface unit 128 also being configured to electrically interface and couple with output transducers 114 configured to transmit sensory output signals associated with a virtual medical-imaging system to the patient 900; and (II) a processor 102 configured to electrically interface and couple to the biometric-information sensors 110, and the output transducers 114; and (III) a memory device 104 electrically coupled to the processor 102, and the memory device 104 tangibly embodying a computer program 106, and the computer program 106 configured to be readable by, and executable by, the processor 102; and the computer program 106 also configured to direct the processor 102 to: (A) transmit, to the patient 900 via the output transducers 114, the sensory output signals associated with the virtual medical-imaging system; and (B) receive, from the patient 900 via of the biometric-information sensors 110, at least one of the biometric-information signals of the patient 900 that represent the biometric reaction of the patient 900 receiving the sensory output signals associated with the virtual medical-imaging system; and (C) transmit, to the patient 900 via the output transducers 114, patient-training information configured to urge the patient 900 to improve a state of calmness of the patient 900. At least some of the patient-training information takes into account at least one of the biometric-information signals received via the biometric-information sensors 110.

In view of the foregoing, there is provided an apparatus, comprising a computer system 100 (a system) including an interface unit 128 being configured to: (I) transmit, to the patient 900, sensory output signals associated with a virtual medical-imaging system; and (II) receive, from the patient 900, biometric-information signals of the patient 900 that represent a biometric-reaction of the patient 900 in response to the patient 900 receiving at least one of the sensory output signals associated with the virtual medical-imaging system; and (III) transmit, to the patient 900, patient-training information configured to urge the patient 900 to improve the state of calmness of the patient 900. At least some of the patient-training information takes into account at least one of the biometric-information signals that were received.

It will be appreciated that the computer system 100 (the system) may be integrated with a real medical-imaging system (if so desired).

There is provided a method of processing signals associated with (A) biometric-information sensors 110 configured to transmit (provide) biometric-information signals of a patient 900 in response to a biometric reaction of the patient 900, and (B) output transducers 114 configured to receive (such as from a computer system 100) and transmit (such as to a patient 900) sensory output signals associated with a virtual medical-imaging system to the patient 900, and the output transducers 114 also configured to receive and transmit patient-training information configured to urge the patient 900 to improve a state of calmness of the patient 900. The method includes (comprises): operation (I) electrically interfacing and coupling with the biometric-information sensors 110; and operation (II) electrically interfacing and coupling with the output transducers 114; and operation (III) transmitting, to the output transducers 114, sensory output signals associated with the virtual medical-imaging system to the patient 900; and operation (IV) receiving, from the biometric-information sensors 110, the biometric-information signals of the patient 900 in response to the biometric reaction of the patient 900 to the sensory output signals associated with the virtual medical-imaging system to the patient 900; and operation (V) transmitting, to the output transducers 114, the patient-training information configured to urge the patient 900 to improve the state of calmness of the patient 900, in which at least some of the patient-training information takes into account at least one of the biometric-information signals that were received.

FIG. 8 depicts a schematic view of an embodiment of a user-interface screen 134 to be presented (via the display device 118, also called an output device) to an operator by the computer system 100 of FIG. 1.

It will be appreciated that the user-interface screen 134 may be varied to include or exclude the items shown on the user-interface screen 134, such as various measurements of the biometric-information signals (heart rate, oxygen level, etc.).

FIG. 9 depicts a schematic view (a flow chart) of a method 800 associated with the operations of the computer system 100 of FIG. 1.

The method 800 is for improving, at least in part, image stability exhibited by a real medical image (known and not depicted) to be generated by a real medical-imaging system (known and not depicted) as a patient 900 (depicted in FIG. 2) remains motionless relative to the real medical-imaging system while the real medical-imaging system generates the real medical image of the patient 900. The method 800 includes and is not limited to (comprises) a transmit operation 802, including: transmitting, to the patient 900 via output transducers 114, (i) sensory output signals being associated with a virtual medical-imaging system, and (ii) patient-training information configured to improve an ability of the patient 900 to remain relatively motionless while the patient 900, in use, receives the sensory output signals. The method 800 also includes a receive operation 804, including: receiving, from the patient 900 via biometric-information sensors 110, biometric-information signals representing biometric reactions of the patient 900 in response to the patient 900, in use, receiving the sensory output signals. The method 800 also includes an adaptation operation 806, including: adapting at least some of the patient-training information based on changes detected in the biometric-information signals received from the patient 900; improvement, at least in part, of the ability of the patient 900 to remain relatively motionless provides improvement, at least in part, to the image stability exhibited by the real medical image to be generated by the real medical-imaging system as the patient 900 remains motionless relative to the real medical-imaging system while the real medical-imaging system generates the real medical image of the patient 900.

In accordance with an optional embodiment, the method 800 is adapted to include an evaluation operation 808, including: evaluating an outcome of the patient-training information provided to the patient 900. The method 800 is further adapted to include a provision operation 810, including: providing a determination indicating whether the image stability exhibited by the real medical image of the patient 900, to be generated by the real medical-imaging system, is suitable for an improved medical diagnosis (wastage of time and effort is reduced, at least in part, for making a relatively more accurate medical diagnosis with improved confidence as a result of the patient 900 utilizing the patient-training information for remaining motionless while the real medical image is generated by the real medical-imaging system).

### Additional Technical Features

Referring to the embodiment as depicted in FIG. 1, the computer system 100 of FIG. 1 is configured to be utilized intra operatively for a surgical session. That is, while the patient 900 is undergoing a surgical treatment, the patient 900 remains conscious during the surgical treatment, and the computer system 100 is utilized by the patient 900 for assisting the patient 900 is remaining as calm as possible during the surgical treatment.

Referring to the embodiment as depicted in FIG. 1, the computer system 100 of FIG. 1, the computer system 100 is configured to receive and/or analyze an additional collection of metrics, such as a motion histogram over time, the sound emission by the patient 900, the heart rate of the patient 900, the blood pressure of the patient and/or an anxiety questionnaire to be administered to the patient 900 (such as the GAD-7 and/or the McMurtry scale). Generalized Anxiety Disorder 7 (GAD-7) is a self-reported questionnaire for screening and severity measuring of generalized anxiety disorder (GAD). GAD-7 has seven items, which measure severity of various signs of GAD according to reported response categories with assigned points. Assessment is indicated by the total score, which made up by adding together the scores for the scale all seven items. The Children's Fear Scale was adapted from the Faces Anxiety Scale to measure fear in children undergoing painful medical procedures. The Children's Fear Scale initial validation study (McMurtry, 2011) with children undergoing venipuncture demonstrated construct validity (through high concurrent validity with another measure of child fear and moderate discriminant validity with child coping behaviour) as well as test-retest and interrater reliability.

Referring to the embodiment as depicted in FIG. 1, the computer system 100 of FIG. 1, the computer system 100 is configured to utilize a convolutional neural network (CNN) algorithm to measure the robust biofeedback metrics and compare to a database of other patients with similar metrics to: (A) predict the imaging quality score (B) instruct the computer system 100 to adapt the audio and/or visual feedback (to the patient 900) for optimal performance, and/or (C) generate a plan for future training which can be performed even at home on a smaller format with less computer integration (i.e., a cardboard version of a virtual reality head set).

Referring to the embodiment as depicted in FIG. 1, the computer system 100 of FIG. 1, the computer system 100 is configured to stop operations by way of an input received from the patient 900 and/or the operator (technician) at any time, preferably with a time stamp included in the metrics.

Referring to the embodiment as depicted in FIG. 1, the computer system 100 of FIG. 1, the computer system 100 is configured to (A) show or display (via the display device 118 to an administrator, operator, etc.) the biofeedback interaction. Preferably, the computer system 100 is configured to seamlessly switch the user experience between: (A) a simulation mode, (B) a training mode, and (C) an anxiety score (preferably from within the virtual reality module). It will be appreciated that any of the above items may be time stamped and recorded.

The following is offered as further description of the embodiments. Any one or more of any technical feature (described in the detailed description, the summary and the claims) may be combinable with any other one or more of any technical feature (described in the detailed description, the summary and the claims). It is understood that each claim in the claims section is an open ended claim unless stated otherwise. Unless otherwise specified, relational terms used in these specifications should be construed to include certain tolerances that the person skilled in the art would recognize as providing equivalent functionality. By way of example, the term perpendicular is not necessarily limited to 90.0 degrees, and may include a variation thereof that the person skilled in the art would recognize as providing equivalent functionality for the purposes described for the relevant member or element. Terms such as “about” and “substantially”, in the context of configuration, relate generally to disposition, location, or configuration that are either exact or sufficiently close to the location, disposition, or configuration of the relevant element to preserve operability of the element within the invention which does not materially modify the invention. Similarly, unless specifically made clear from its context, numerical values should be construed to include certain tolerances that the person skilled in the art would recognize as having negligible importance as they do not materially change the operability of the invention. It will be appreciated that the description and/or drawings identify and describe embodiments of the apparatus (either explicitly or inherently). The apparatus may include any suitable combination and/or permutation of the technical features as identified in the detailed description, as may be required and/or desired to suit a particular technical purpose and/or technical function. It will be appreciated that, where possible and suitable, any one or more of the technical features of the apparatus may be combined with any other one or more of the technical features of the apparatus (in any combination and/or permutation). It will be appreciated that persons skilled in the art would know that the technical features of each embodiment may be deployed (where possible) in other embodiments even if not expressly stated as such above. It will be appreciated that persons skilled in the art would know that other options would be possible for the configuration of the components of the apparatus to adjust to manufacturing requirements and still remain within the scope as described in at least one or more of the claims. This written description provides embodiments, including the best mode, and also enables the person skilled in the art to make and use the embodiments. The patentable scope may be defined by the claims. The written description and/or drawings may help to understand the scope of the claims. It is believed that all the crucial aspects of the disclosed subject matter have been provided in this document. It is understood, for this document, that the word “includes” is equivalent to the word “comprising” in that both words are used to signify an open-ended listing of assemblies, components, parts, etc. The term “comprising”, which is synonymous with the terms “including,” “containing,” or “characterized by,” is inclusive or open-ended and does not exclude additional, unrecited elements or method steps. Comprising (comprised of) is an “open” phrase and allows coverage of technologies that employ additional, unrecited elements. When used in a claim, the word “comprising” is the transitory verb (transitional term) that separates the preamble of the claim from the technical features of the invention. The foregoing has outlined the non-limiting embodiments (examples). The description is made for particular non-limiting embodiments (examples). It is understood that the non-limiting embodiments are merely illustrative as examples.

What is claimed is:

1. An apparatus, comprising:
a system including an interface unit configured to:
transmit, to a patient, sensory output signals associated with a virtual medical-imaging system; and
receive, from the patient, biometric-information signals of the patient that represent a biometric-reaction of the patient in response to the patient receiving at least one of the sensory output signals associated with the virtual medical-imaging system; and
transmit, to the patient, patient-training information configured to urge the patient to improve a state of calmness of the patient, and at least some of the patient-training information takes into account at least one of the biometric-information signals that was received.

2. An apparatus, comprising:
a system being positionable relative to a patient, and the system being configured to:
electrically connect to output transducers, in which the patient is positionable proximate to the output transducers, and the output transducers are configured to transmit, to the patient, sensory output signals associated with a virtual medical-imaging system; and
electrically connect to biometric-information sensors, in which the patient is positionable proximate to the biometric-information sensors, and the biometric-information sensors are configured to receive biometric-information signals from the patient; and
transmit to the patient via the output transducers:
the sensory output signals associated with the virtual medical-imaging system; and
patient-training information configured to improve an ability of the patient to remain relatively motionless while the patient, in use, receives the sensory output signals; and
receive the biometric-information signals from the patient via the biometric-information sensors, in which the biometric-information signals represent biometric reactions of the patient in response to the patient, in use, receiving, and reacting to, the sensory output signals; and
adapt at least some of the patient-training information based on changes detected in the biometric-information signals received from the patient; and
whereby improvement, at least in part, of the ability of the patient to remain relatively motionless provides improvement, at least in part, to image stability exhibited by a real medical image to be generated by a real medical-imaging system as the patient remains motionless relative to the real medical-imaging system while the real medical-imaging system generates the real medical image of the patient.

3. The apparatus of claim 2, wherein:
the system is also configured to evaluate an outcome of the patient-training information provided to the patient.

4. The apparatus of claim 3, wherein:
the system is also configured to provide a determination indicating whether the image stability exhibited by the real medical image of the patient, to be generated by the real medical-imaging system, is suitable for an improved medical diagnosis; and
whereby wastage of time and effort is reduced, at least in part, for making a relatively more accurate medical diagnosis with improved confidence as a result of the patient utilizing the patient-training information for remaining motionless while the real medical image is generated by the real medical-imaging system.

5. The apparatus of claim 2, wherein:
at least one of the biometric-information sensors is selected from the group consisting of a heart rate sensor, a blood pressure sensor, an oxygen sensor, and a microphone.

6. The apparatus of claim 2, wherein:
at least one of the output transducers is selected from the group consisting of a speaker, a display device, and a haptic device.

7. The apparatus of claim 2, wherein:
the patient-training information includes audio instructions for suggested breathing patterns, visualization techniques, and verbal suggestions.

8. The apparatus of claim 2, wherein:
the patient-training information is configured to suggest, to the patient, various cognitive strategies in order to achieve a preferred bodily state while the output transducers continue to transmit to the patient.

9. The apparatus of claim 2, wherein:
the system is also configured to detect any changes in physiological information as provided by the biometric-information sensors; and
the system is also configured to determine whether the patient has remained motionless for a predetermined period of time based on the biometric-information signals provided by the biometric-information sensors; and
the system is also configured to change the sensory output signals to be sent to the patient via the output transducers; and
the system is also configured to transmit the change in the sensory output signals to the patient, via the output transducers, for a case where a determination has been made that the patient has remained motionless for the predetermined period of time.

10. The apparatus of claim 2, wherein:
the system is also configured to compute an objective score indication that indicates a degree of a potential image quality that may be related to a hypothetical medical image to be generated by the real medical-imaging system for a case where the patient-training information is no longer provided to the patient, and the real medical-imaging system was to be hypothetically used for generating the real medical image of the patient at a current level of training that the patient has received from the patient-training information.

11. The apparatus of claim 2, wherein:
the system is also configured to compute whether the patient may require sedation during a medical procedure, in view of accumulated training provided to the patient and in view of the improvement made by the patient during training.

12. The apparatus of claim 2, wherein:
the system is also configured to compute whether the patient is ready to experience the real medical-imaging system with an improved confidence in a potential image quality resulting from the real medical image that may be generated by the real medical-imaging system in view of accumulated training provided to the patient.

13. The apparatus of claim 2, wherein:
the system is also configured to compute an estimation that indicates an amount of additional stimulation-training time needed by the patient in order to improve a potential medical-image quality to be generated by the real medical-imaging system.

14. The apparatus of claim 2, wherein:
the system is also configured to transmit a game, via the output transducers, to the patient.

15. The apparatus of claim 2, wherein:
the system is also configured to provide an assessment indication of an assessment of a predicted image quality of the real medical image to be generated by the real medical-imaging system.

16. The apparatus of claim 15, wherein:
the assessment indication tracks a change in an expected image quality score for the patient.

17. The apparatus of claim 2, wherein:
the system is also configured to compute an estimate of a potential quality of the real medical image to be generated by the real medical-imaging system based on the patient-training information provided to the patient and based on progress made by the patient while the patient-training information was provided to the patient.

18. The apparatus of claim 2, wherein:
the system is also configured to transmit information, via at least one of the output transducers to the patient, in which the information helps to maintain attention of the patient in an occupied and focused state while the patient ignores, at least in part, unwanted stimulation that may interfere with attention of the patient during a training session.

19. A method to improve, at least in part, image stability exhibited by a real medical image to be generated by a real medical-imaging system as a patient remains motionless relative to the real medical-imaging system while the real medical-imaging system generates the real medical image of the patient, and the method comprising:
transmitting, to the patient via output transducers, sensory output signals being associated with a virtual medical-imaging system, and patient-training information configured to improve an ability of the patient to remain relatively motionless while the patient, in use, receives the sensory output signals; and
receiving, from the patient via biometric-information sensors, biometric-information signals representing biometric reactions of the patient in response to the patient, in use, receiving the sensory output signals; and
adapting at least some of the patient-training information based on changes detected in the biometric-information signals received from the patient; and
whereby improvement, at least in part, of the ability of the patient to remain relatively motionless provides improvement, at least in part, to the image stability exhibited by the real medical image to be generated by the real medical-imaging system as the patient remains motionless relative to the real medical-imaging system while the real medical-imaging system generates the real medical image of the patient.

20. The method of claim 19, further comprising:
evaluating an outcome of the patient-training information provided to the patient; and
providing a determination indicating whether the image stability exhibited by the real medical image of the patient, to be generated by the real medical-imaging system, is suitable for an improved medical diagnosis; and
whereby wastage of time and effort is reduced, at least in part, for making a relatively more accurate medical diagnosis with improved confidence as a result of the patient utilizing the patient-training information for remaining motionless while the real medical image is generated by the real medical-imaging system.
